(19) United States
(12) Patent Application Publication
(10) Pub. No.: US 2007/0185094 A1
(43) Pub. Date: Aug. 9, 2007

(54) Novel 3-substitued-1,4-benzodiazepines

(75) Inventors: Eric Lattmann, Birmingham (GB); Michael Offel, Victoria (AU); Harjit Singh, Dudley (GB)

Correspondence Address: SYNNESTVEDT LECHNER & WOODBRIDGE LLP, P O BOX 592, 112 NASSAU STREET, PRINCETON, NJ, 08542-0592, US

(73) Assignee: ASTON UNIVERSITY, BIRMINGHAM (GB)

(21) Appl. No.: 10/558,786

(22) PCT Filed: May 27, 2004
(86) PCT No.: PCT/GB04/02252

§ 371 Date: Sep. 26, 2006

(30) Foreign Application Priority Data
May 30, 2003 (GB) 0312365.0

(51) Int. Cl. A61K 31/551; C07D 243/14

(52) U.S. Cl. 514/221; 540/504

(57) ABSTRACT

The present invention relates to compounds of formula (I). The invention also relates to methods for preparing the compounds and their uses as CCK receptor ligands and CCK antagonists.

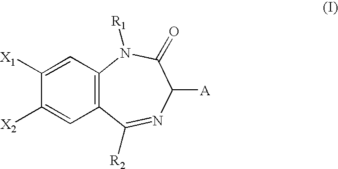

The present invention relates to novel 3-substituted-anilino-1,4-benzodiazepines, their preparation and their use as non-peptide CCK ligands, particularly in pharmaceutical formulations thereof.

Cholecystokinins (CCKs) act as anti-opioid peptides. CCK was initially described as a regulatory hormone found in endocrine cells of the gastro-intestinal (GI) tract. Some CCKs share a common amino acid sequence with gastrin, which is involved in control of gastric acid and pepsin secretion. CCK's have also been found throughout the central nervous system (CNS), where they are believed to act as neurotransmitters and/or modulators of many important functions. There are various known structures of CCK, identified with reference to the number of amino acids they comprise. For example, CCK-8 is a naturally-occurring predominating CCK peptide and, having only eight amino acids, is the minimum fully-active sequence, although small amounts of CCK-4 may also be present.

Cholecystokinin (CCK) plays an important role in the invasiveness and the production of matrix metalloproteinase-9 (MMP-9) in human pancreatic cancer cell lines. The pathway of the invasiveness may be associated with MMP-9 of those lines regulated by CCK.

Cholecystokinin (CCK) receptors play a role in the development and growth of pancreatic cancers. The gut hormone cholecystokinin exerts various actions on the gastrointestinal tract, including the regulation of growth. The hormone has been reported to induce hypertrophy and hyperplasia of the pancreas and to enhance chemically-induced pancreatic carcinogenesis in animals. Stimulation of endogenous cholecystokinin secretion through the induction of deficiency of intraintestinal proteases and bile salts by trypsin-inhibiting nutrients, bile salt-binding drugs or surgical intervention is also capable of stimulating growth and tumour development in the rat. In man, factors suggested to increase the risk of pancreatic cancer, such as a high-fat and high-protein diet or gastrectomy, are known to stimulate plasma cholecystokinin secretion. Receptors for cholecystokinin have been demonstrated on human pancreatic adenocarcinomas, and cholecystokinin has been demonstrated to enhance the growth of xenografted pancreatic cancer and to inhibit growth of gastric and bile duct cancer.

There are two subtypes of CCK receptor which were initially termed as type-A and type-B, reflecting their preferential localisation in the alimentary tract and in the brain, respectively. Recently, these receptors have been re-named as CCK1 and CCK2, respectively, although the original designation is used hereinbelow with respect to the present invention. The molecular cloning of two CCK receptor subtypes, one from rat and human pancreas and one from human brain, has confined the pharmacological classification of CCK receptors. Both CCK1 and CCK2 receptors belong to the family of G-protein coupled receptors. However, the differential distribution of CCK1 and CCK2 receptors in the peripheral vs. central nervous system is not absolute, and CCK1 receptors have been shown to be present in discrete regions of the CNS, including the spinal cord, particularly in primates.

The functions of the CCK1 receptors in the brain are poorly understood, whereas the CCK2 receptor is known to mediate anxiety, panic attacks, satiety and pain. Therefore, antagonists to CCK and to gastrin have been useful for preventing and treating CCK-related and/or gastrin-related disorders of the GI and CNS of animals, especially of humans. Just as there is some overlap in the biological activities of CCK and gastrin, antagonists also tend to have affinity for both receptors. In a practical sense, however, there is enough selectivity for the respective receptors that greater activity against specific CCK- or gastrin-related disorders can often also be identified.

Selective CCK antagonists are themselves useful in treating CCK-related disorders of the appetite regulatory systems of animals as well as in potentiating and prolonging opiate-mediated analgesia, thus having utility in the treatment of pain, while selective gastrin antagonists are useful in the modulation of CNS behaviour, as a palliative for gastrointestinal neoplasms, and in the treatment and prevention of gastrin-related disorders of the GI system in humans and animals, such as peptic ulcers, Zollinger-Ellison syndrome, antral G cell hyperplasia and other conditions in which reduced gastrin activity is of therapeutic value. Also, since CCK and gastrin also have trophic effects on certain tumours, antagonists of CCK and gastrin are useful in treating these tumours.

Various chemical classes of CCK-receptor antagonists have been reported. These include pyrazolidinones showing good selectivity for CCK_{B} receptors (Howbert, J. J. et. al.; Diphenylpyrazolidinone and benzodiazepine cholecystokinin antagonists: A case of convergent evolution in medicinal chemistry., Bioorg. Med. Chem. Lett. 1993, 3, 875-880.), ureidoacetamides which are potent and selective ligands for CCK_{B}/gastrin receptors (WO 91/113874), ureidophenoxyacetanilides (Takeda, Y. et. al.; Synthesis of phenoxyacetic acid derivatives as highly potent antagonists of gastrin/cholecystokinin-B receptors, Chem. Pharm Bull. 1998, 46, 951-961), ureidomethylcarbamoylphenylketones (Hagishita, S.; et. al., Ureido-methylcarbamoyl-phenylketones as selective CCK_{B} receptor antagonists. Bioorg. Med. Chem. 1997, 5, 1695-1714), and ureidobenzodiazepine derivatives (Evans, B. E.; et. al., Design of potent, orally effective, non peptidal antagonists of the peptide hormone cholecystokinin, Proc. Natl. Acad Sci. USA 1986, 83, 4918-4922).

Benzodiazepine Derivatives

Benzodiazepines were very weak in displacing CCK in mouse brain (IC₅₀=10 μM)¹. In a study from Japan anthramycin², a benzodiazepine derivative, was reported to be a potent antagonists of CCK in mice. Anthramycin reversed CCK-8 induced satiety and was shown to displace [¹²⁵I] CCK-8 binding in different brain regions, especially in the cortex. Further investigations are underway to elucidate the pharmacological potential of this compound.

Asperlicin represented a major advance in the development of CCK receptor antagonists. It demonstrated 300-400 times more affinity for pancreatic and gallbladder CCK receptors than proglumide. However, this compound demonstrated poor stability and poor oral bioavailability³. By combining the elements of Asperlicin, L-364,286 was the first successful synthetic analogue, in which the diazepam-like structure is linked with a 3-amido group.

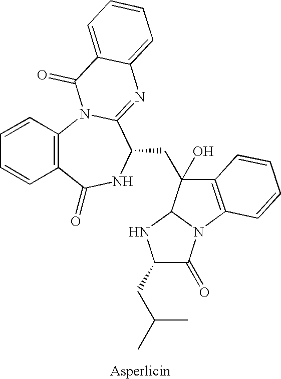

New efforts to optimise the CCK_{A} antagonist activity of these benzodiazepine derivatives led to devazepide (MK-329, formerly L-364,718) (Panel 1) an extremely potent and orally active CCK_{A} antagonist (IC₅₀=0.1 nM inhibition of ¹²⁵I-CCK-8 rat pancreas binding). This compound had a more than 1000-fold selectivity for the CCK_{A} receptor and a longer lasting efficacy.

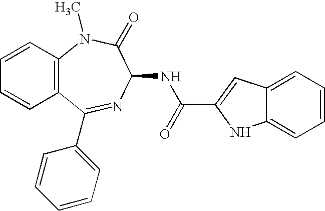

### Panel 1. 3-Amido-1,4-benzodiazepine derivative L-364,718/MK-329/Devazepide

Devazepide possessed a potent CCK_{A} blocking activity in different tissues⁴. The pancreatic amylase secretion was antagonised with a 2,000,000 times higher potency than proglumide. Devazepide has been claimed⁵ to be a selective antagonist inhibiting the effects of CCK-8 (Sincalide) on food intake. In contrast, when CCK-8, was secreted from the gastric mucosa, the release of both bile from the gallbladder, and the release of digestive enzymes from the pancreas were stimulated⁶. Devazepide was a key tool in the autoradiographical demonstration of the presence of CCK_{A} receptors in the various regions of the brain⁷. During the extensive development of L-364, 718 it was noted that some analogues lost their selectivity for CCK_{A}.

Devazepide in the Treatment of Cancer

Devazepide inhibited in vitro the proliferation of cells and induced morphologic changes in the mucous-secreting, autonomously proliferating human cancer colon cell line (HT29-S-B6). Addition of Devazepide (10 μM) for at least 3 days in the exponential phase of growth enhanced the baseline production of gastric M1 mucins 2-3-fold and that of carcinoembryonic antigens 5-fold. Moreover, devazepide induced an increase in the amount of the MUC-5AC mRNA expressed by HT29-S-B6 cells. The increase in mucins secretion, induced by devazepide, was persistent after removal and independent of the presence of serum⁸. Devazepide inhibited the growth of CCK receptor-positive human pancreatic cancer in athymic mice. Based on these activities and the ability of Devazepide to transiently increase food intake and to enhance morphine analgesia in murine models, an open trial⁹ of Devazepide was conducted in 18 patients with advanced pancreatic cancer in whom the CCK receptor status of the tumors was unknown. Tumor response, pain control, and nutritional parameters (hunger rating, caloric intake, body weight, and anthropometrics) were serially assessed. The results of the study failed to demonstrate any impact of Devazepide on tumor progression, pain, or nutrition. Toxicity was mild and limited to nausea, vomiting, diarrhea, and abdominal cramps, with 17 of 18 patients able to tolerate treatment.

Ureidobenzodiazepine Derivatives

When the 3-amido linkage was replaced with a benzamido urea, the CCK_{A} affinity decreased and the CCK_{B} affinity increased substantially. The most interesting compound developed by Merck scientists was L-365,260 (Panel 2). L-365,260 showed a high affinity for CCK_{B} receptors in rats, mice and in humans. Devazepide was reported to have a 125 fold greater affinity for pancreatic CCK_{A} receptors, than for gastrin receptors. L-365,260 has shown only an 80 fold grater affinity for gastrin/CCK_{B} receptors than for pancreatic CCK_{A}.

Both Devazepide and L-365,260 were investigated¹⁰ as to whether the satiety response to CCK is mediated by CCK_{A} or CCK_{B} receptors. L-365, 260 was reported to be 100 times more potent than Devazepide in increasing feeding frequency and preventing satiated rats. The conclusion from the study was that endogenous CCK causes satiety by interaction with CCK_{B} receptors in the brain.

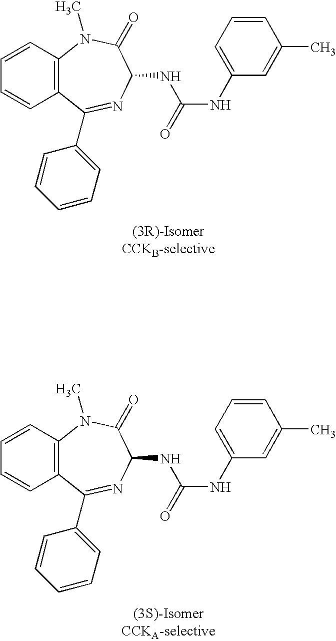

### Panel 2: Isomers of 3-ureido-1,4-benzodiazepine derivative L-365,260

The high affinity CCK_{B}-selective urea L-365,260 and related analogues is dependent upon the stereochemistry at the C-3 position of the benzodiazepine ring, the (3S)-enantiomer generally being CCK_{A} selective and the (3R)-isomer CCK_{B} selective. L-365,260 shows high affinity for CCK_{B} receptors in rats, mice and in humans. Although L-365,260 represents a benzodiazepine structure, it has no affinity to GABA-A receptors and does not induce tolerance and withdrawal in animal models. During phase 1 clinical trials it was found that L-365,260 had a limited oral bioavailability due to its low aqueous solubility and bio-distribution studies in mice¹¹ have shown very low brain uptakes (<0.8% dose/gram) after intravenous injections.

L-365,260 and its Role in Cancer

The cell line LN 36 responded in vitro¹² with an increased cell number to stimulation by gastrin-17 and decreased cell number to inhibition by the CCK-B receptor antagonist L-365,260. Specific cholecystokinin (CCK) receptor and gastrin receptor antagonists were used to assess what role, if any, these receptors have in autocrine cell growth. Although the cholecystokinin receptor antagonist, Devazepide, inhibited cell proliferation in a broad spectrum of cell lines, the gastrin antagonist, Devazepide, had no effect on cell proliferation. In addition neither added gastrin 17, nor sulfated cholecystokinin 8, could reverse the inhibitory action of Devazepide. It is proposed that Devazepide inhibits cell proliferation independently of classical gastrin/CCK receptors¹³.

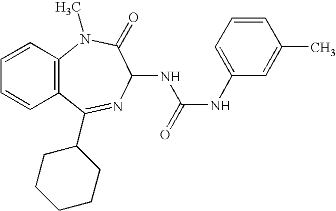

### Panel 3. L-708,474

One of the most potent and selective CCK_{B} receptor ligand is L-708,474 (Panel 3). L-708,474 displayed a thirty-fold higher affinity than L-365,260 (IC₅₀=8.5 nM) at the CCK_{B} receptor and was found markedly more selective for CCK_{B} receptors over CCK_{A} (6,500-fold v. 87-fold). The enhanced binding affinities of the 5-cyclohexyl benzodiazepines demonstrated the importance of the size of the lipophilic substituent at the C-5 position of the benzodiazepine template. L-708,474 (IC₅₀=0.28 nM) was an exceptionally high affinity ligand at the CCK_{B} receptor. L-708,474 is considerably more potent than either the cyclopentyl (IC₅₀=16 nM) or the cyclobutyl (IC₅₀=29.9 nM) analogues. It has shown an increased lipophilicity in comparison to L-365,260, enhanced potency and selectivity for the CCK_{B} receptor, but a decreased bioavailability.

Based on Merck's phase 1 trials with L-365,260 a second generation of CCK_{B}/gastrin receptor antagonists was developed. The chemists at Merck hoped to increase the oral bioavailability of the newly synthesized compounds by introducing groups with water-solublising properties.

One of the compounds with an increased bioavailability¹⁴, L-740,093 (panel 4), containing a basic amidine structure, was found to be extremely potent. L-740,093 showed a one hundred fold improved water solubility as the HCl salt compared to L-365,260. L-740,093 displayed an IC₅₀ of 0.1 nM for the CCK_{B} receptor and had a CCK_{A}/CCK_{B} ratio of approximately 16060. Thus L-740,093 seems to be suitable for oral treatment in humans.

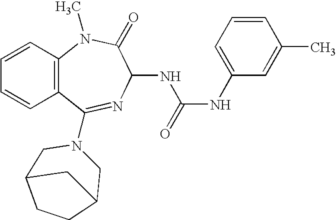

### Panel 4: 3-Ureido-1,4-benzodiazepine derivative L-740,093

Another approach to increase the water solubility of L-365,260 in order to achieve good levels of oral bioavailability, was successfully performed by incorporating acidic solubilising groups into the 3-phenyl ring of the acylurea moiety¹⁵.

The C5-cyclohexyl derivatives incorporating aminotetrazole group (L-737,425, Panel 5)) was the most potent and selective (CCK_{A}/CCK_{B}=37000) antagonists so far reported for CCK-B/gastrin receptors. However, the preparation of this compound includes a synthetic complexity.

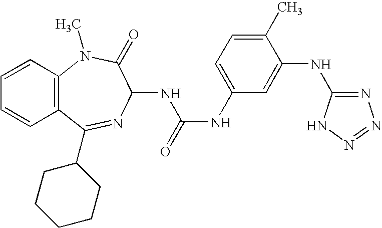

### Panel 5: 3-Ureido-1,4-benzodiazepine derivative L-737,425

A novel series of 1-aroylmethyl analogues of L-365,260 was prepared and evaluated for activity as CCK_{B}/gastrin receptor antagonists by the Yamanouchi group. YM022 (Panel 6) has shown to be a significantly more potent antagonists of pentagastrin than L-365,260. YM022 exhibited a very high CCK_{B}/gastrin receptor affinity (IC₅₀=0.11 nM) and a CCK_{A}/CCK_{B} ratio about 1300¹⁶. YM022 showed, compared to L-365,260, a better bioavailability and is a compromise between the lipophilicity and selectivity for the CCK_{B} receptor. However, the improvement in the obtained potency did not compensate the increase in synthetic complexity.

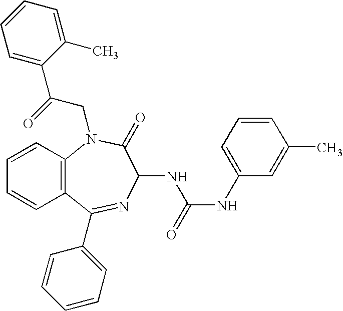

### Panel 6: 1-Benzoylmethyl 3-ureido-1,4-benzodiazepine derivative YM022

The antiproliferative potency of YM022 was evaluated by using N-hCCKBR cells. YM022 had the most potent activities in competing with [¹²⁵I]CCK-8 or [¹²⁵I]gastrin I binding, inhibition of CCK-8- or gastrin I-induced phosphoinositide hydrolysis and increasing cytoplasmic free calcium. Interestingly, a potent antagonist for rat CCK-B/gastrin receptors did not have such activities in N-hCCKBR cells. YM022 inhibited the CCK-8- or gastrin I-induced [methyl-3H]thymidine incorporation of N-hCCKBR cells in a dose-dependent manner. In the absence of exogenous peptide ligands, YM022 also inhibited the proliferation of several human cancer cell lines expressing the genes for both gastrin and its receptor. These results suggest that YM022 could intervene in the autocrine stimulation of human tumor cell lines through CCK-B/gastrin receptors. N-hCCKBR cells are an excellent tool to screen for novel human CCK-B/gastrin receptor antagonists possessing antiproliferative activity for human cancer cells¹⁷.

Potentiation of Clinical Effects

It was reported that the cholecystokinin antagonist Proglumide potentiated morphine analgesia The effect of Proglumide on spinal and supraspinal mu and spinal delta analgesia were investigated in mice in order to understand more fully the opiate receptor subtypes involved with this effect. It was found that Proglumide alone had no effect on tailflick latencies, but increased, in a dose-dependent manner, tailflick latencies in morphine-tolerant mice. Proglumide also potentiated morphine analgesia in naive mice in a dose-dependent manner, with a maximal effect at 5-10 mg/kg. It both shifted the dose-response curve for morphine analgesia to the left and prolonged morphine's duration of action. Proglumide increased the sensitivity of supraspinal mu 1 receptor mechanisms of analgesia without influencing spinal mechanisms. Proglumide administered subcutaneously potentiated the analgesic actions of intracerebroventricular [D-Ala2,MePhe4,Gly(ol)5]enkephalin (DAGO; (mu 1), but not intrathecal DAGO (mu 2) or [D-Pen2,D-Pen5]enkephalin (DPDPE; delta). The selective mu 1 receptor antagonist naloxonazine blocked proglumide-enhanced morphine analgesia¹⁸.

As CCK receptors are present on pancreatic carcinoma cells it was determined whether either CCK itself or an antagonist of CCK could modulate the sensitivity of the human pancreatic cell line MIA-PaCa2 to cisplatin (DDP). The IC₅₀ for a 1-h exposure to DDP was 35.3±3.2 (SD) μM. Exposure to CCK₈ octapeptide at physiologic and supra-physiologic concentrations did not alter the sensitivity of MIA-PaCa2 cells to DDP. The CCK receptor antagonist Devazepide was directly cytotoxic to the MIA-PaCa2 cells on a constant exposure schedule with an IC50 of 9.5±1.4 (SD) μM. Devazepide enhanced the sensitivity of MIA-PaCa2 cells to DDP by a factor of 3.5 and the interaction between DDP and Devazepide was shown to be synergistic by median-effect analysis. At a level of 50% cell kill, the combination index was 0.58±0.10. The ability of Devazepide to sensitize cells to DDP was schedule-dependent and required prolonged exposure to the antagonist following a 1-h exposure to DDP¹⁹.

¹ Sugaya, K.; Matsuda, I.; Uruna, T. and Kubota, K., Studies on the CCK antagonism by benzodiazepines: Displacement of CCK by benzodiazepines in the binding in mouse brain CCK receptor. Jpn. J. Pharmacol. 1984, 40, 114 p.

² Kubota, K.; Sugaya, K.; Koizumi, Y. and Toda, M., Cholecystokinin antagonism by anthramycin in the central nervous system in mice. Brain Res. 1989, 485, 62-66.

³ Bock, M. G.; DiPardo, R. M.; Rittle, K. E., et al., Cholecystokinin antagonists. Synthesis of asperlicin analogues with improved potency and water solubility, J. Med. Chem. 1986, 29, 1941-1952.

⁴ Hosontanti, R.; Chowdhury, P.; McKay, D. and Rayford, P. L., (1988), Effect of L-364,718, a new CCK antagonist, on amylase scretion in isolated rat pancreatic acini., Pancreas, 3, 95-98. Anderson, L. and Dockray, G. L., The cholecystokinin antagonist L-364,718 inhibits the action of cholecystokinin but not bombesin on rat pancreatic secretion in vivo., Euro. J. Pharmacol. 1988, 146, 307-311.

⁵ Silver, A. J.; Flood, J. F.; Song, A. M. and Morley, J. E., Evidence for a physiological role for CCK in the regulation of food intake in mice., Am. J. Physiol. 1989, 256, 646-652.

⁶ Dunlop, J.; CCK receptor antagonists, Gen Pharmacol. 1998, 31, 519-524.

⁷ Hill, D. R.; Shaw, T. M.; Graham, W. and Woodruff, G. N., Autoradiographical detection of cholecystokinin-A receptors in primate brain using ¹²⁵I bolton Hunter CCK-8 and 3H-MK-329. J. Neurosci. 1989, 10, 1070-1081.

⁸ Forgue-Lafitte, M. E., Coudray, A. M., Aubert, J. P., Gespach, C., Bara, J. Devazepide (L-364718) inhibits growth and increases expression of tumor markers in HT29-S-B6 cells. Acad. Sci. III 1996, 319, 1133-8.

⁹ Abbruzzese J L, Gholson C F, Daugherty K, Larson E, DuBrow R, Berlin R, Levin B. A pilot clinical trial of the cholecystokinin receptor antagonist MK-329 in patients with advanced pancreatic cancer. Pancreas 1992, 7, 165-71.

¹⁰ Dourish, C. T.; Rycroft, W. and Iversen, S. D., Postponement of satiety by blockade of brain cholecystokinin-B receptors., Science 1989, 245, 1509-1511.

¹¹ Haradahira, T.; Inoue, O.; Kobayashi, K. and Suzuki, K., Synthesis and evaluation of 11C-labeled nonpeptide antagonists for cholecystokinin receptors: [11C] L-365,260 and [11C]L-365,346., Nucl. Med. Biol. 1998, 25, 203-208.

¹² Ohlsson B, Fredaong N, Axelson J. The effect of bombesin, cholecystokinin, gastrin, and their antagonists on proliferation of pancreatic cancer cell lines. Scand J Gastroenterol 1999, 34, 1224-9.

¹³ Thumwood, C. M., Hong, J., Baldwin, G. S. Inhibition of cell proliferation by the cholecystokinin antagonist L-364,718. Exp. Cell Res. 1991, 92, 189-92.

¹⁴ Showell, G. A.; Bourrain, S.; Neduvelil, J. G.; Fletcher, A. E.; Baker, R.; Watt, A. P.; Fletcher, A. E.; Freedman, S. P.; Kemp, J. A.; Marshall, G. R.; Patel, S.; Smith, A. J. and Matassa, V. G., High affinity and potent, water soluble 5-amino-1,4-benzodiazepine CCK_{B}/gastrin receptor antagonists containing a cationic solubilizing group, J. Med. Chem. 1994, 37, 719-721.

Castro, L. C.; Broughton, H. B.; Russell, M. G. N.; Rathbone, R. and Watt, A. P., 5-(Piperidin-2-yl)- and 5-(homopiperidin-2-yl)-1,4-benzodiazepines: High-affinity, basic ligands for the cholecystokinin-B receptor. J. Med. Chem. 1997, 40, 2491-2501.

¹⁵ Bock, M. G.; DiPardo, R. M.; Mellin, E. C. and Newton, N. C., Second-generation benzodiazepine CCK-B antagonists. Development of subnanomolar analogues with selectivity and water solubility. J. Med. Chem. 1994, 37, 722-724.

¹⁶ Masato, S.; Yutaka, K.; Yoshinori, O.; Akito, N. and Keiji, M., New 1,4-benzodiazepin-2-one derivatives as gastrin/cholecystokinin-B antagonists., Chem. Pharm. Bull. 1995, 43, 2159-2167.

¹⁷ Murayama, T., Matsumori, Y., Iwata, N., Ito, M., Taniguchi, T., Chihara, K., Matsui, T. Antiproliferative effect of a novel cholecystokinin-B/gastrin receptor antagonist, YM022. Jpn. J. Cancer Res. 1996, 87, 743-50.

¹⁸ Bodnar, R. J., Paul, D., Pasternak, G. W. Proglumide selectively potentiates supraspinal mu 1 opioid analgesia in mice. Neuropharmacol. 1990, 29, 507-10.

¹⁹ Jamshidipour, R., Pinho, E. B., Hom, D. K., Howell, S. B. Enhancement of the cytotoxicity of cisplatin by the cholecystokinin antagonist MK-329 in a human pancreatic cancer cell line. Cancer Chemother. Pharmacol. 1994, 34, 484-90.

It is an object of the present invention to provide novel 3-substituted-anilino-1,4-benzodiazepines. Further objects relate to the, biological activity of said derivatives, particularly, but not exclusively their use as CCK-receptor ligands.

According to a first aspect of the present invention, there is provided a compound of formula (I)

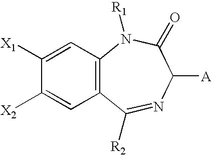

wherein

each of X₁, X₂, and R₂ is independently selected from hydrogen, a halogen, a substituted or unsubstituted cyclic and heterocyclic moiety, substituted or unsubstituted, linear or branched alkyl, alkyloxy, alkylcarbonyl, alkyloxycarbonyl, alkenyl, alkenyloxy, alkenylcarbonyl, alkenyloxycarbonyl, alkynyl, alkynyloxy, alkynylcarbonyl, alkynyloxycarbonyl, aryl, benzyl, arlyoxy, arylcarbonyl, aryloxycarbonyl and sulphur equivalents of said oxy, carbonyl and oxycarbonyl moieties, and a nitrogen containing functional group,

R₁ is selected from hydrogen, a halogen, a substituted or unsubstituted cyclic and heterocyclic moiety, substituted or unsubstituted, linear or branched alkyl, alkylcarbonyl, alkyloxycarbonyl, alkenyl, alkenylcarbonyl, alkenyloxycarbonyl, alkynyl, alkynylcarbonyl, alkynyloxycarbonyl, aryl, benzyl, arylcarbonyl, aryloxycarbonyl and sulphur equivalents of said, carbonyl and oxycarbonyl moieties and

A is selected from hydrogen, hydroxyl, a halogen, a nitrogen-containing heterocycle linked to the diazepine moiety via nitrogen and

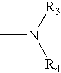

wherein R₃ and R₄ are independently selected from hydrogen, a halogen, a substituted or unsubstituted cyclic and heterocyclic moiety, substituted or unsubstituted, linear or branched alkyl, alkylcarbonyl, alkyloxycarbonyl, alkenyl, alkenylcarbonyl, alkenyloxycarbonyl, alkynyl, alkynylcarbonyl, alkynyloxycarbonyl, aryl, benzyl, arylcarbonyl, aryloxycarbonyl and sulphur equivalents of said, carbonyl and oxycarbonyl moieties and

wherein if A=OH, then R₁ is selected from a substituted or unsubstituted cyclic and heterocyclic moiety, substituted or unsubstituted, linear or branched alkyl, alkylcarbonyl, alkyloxycarbonyl, alkenyl, alkenylcarbonyl, alkenyloxycarbonyl, alkynyl, alkynylcarbonyl, alkynyloxycarbonyl, aryl, benzyl, arylcarbonyl, aryloxycarbonyl and sulphur equivalents of said, carbonyl and oxycarbonyl moieties

Preferably said alkyl-containing moieties (e.g. alkyl, alkyloxycarbonyl etc.) are C₁-C₁₂, more preferably C₁-C₆ and most preferably C₁ to C₄.

Preferably said alkenyl- and said alkynyl-containing moieties are C₂-C₁₂, more preferably C₂-C₆ and most preferably C₂ to C₄.

Preferably, said aryl moiety is substituted or unsubstituted phenyl, napthyl or indolyl. Particularly preferred are m-substituted phenyl, indol-2-yl and indol-3-yl.

Examples of suitable substituents for said heterocyclic, alkyl, alkenyl, alkynyl and aryl moieties include halo, amino, nitro, hydroxy, alkoxy (eg. methoxy) and cyano moieties.

Preferably, said heterocyclic moiety is a monocyclic or bicyclic ring comprising at least one of oxygen, sulphur and nitrogen. Preferably each ring of the heterocyclic moiety is a 3 to 7 membered ring.

Preferably, said cyclic alkyl moiety is a 3 to 7 membered ring and said cyclic alkenyl and alkynyl moieties are preferably, 4 to 7 membered rings. Particularly preferred is cyclohexyl.

Preferably, X₁ and X₂ are independently selected from hydrogen, C₁₋₄ alkyl, halogen, nitro, amino and C₁₋₄ alkoxy.

Preferably, R₁ is selected from hydrogen, C₁₋₄ alkyl, benzyl, alkylcarbonyl, alkyloxycarbonyl, arylcarbonyl, alkenyl, alkynyl alkylcarbonylmethyl, arylcarbonylmethyl and morpholinylalkyl. Particularly preferred are phenylmethyl, ^{t}butylcarbonyl, propargyl, allyl, C₁₋₄ alkyloxycarbonyl, phenylcarbonylmethyl and morpholinyl C₁₋₄ alkyl.

Preferably, R₂ is phenyl or cyclohexyl.

Where A is a nitrogen-containing heterocycle, it is preferably selected from morpholinyl, pyrazolyl, piperazinyl, piperidinyl, quinolinyl, 3,4-dihydroquinolin-1(2H)-yl, and indolyl all of which may be substituted or unsubstituted.

Where A is N(R₃)R₄, R₃ and R₄ are preferably independently selected from hydrogen, C₁₋₄ alkyl, (CH₂)ₙC₁₋₆ alkyl, (CH₂)ₙC₃₋₆ cycloalkyl, pyrenyl, tetrahydronaphthyl, morpholinyl, 1-phenyl-pyrazol-2-yl, tetrahydroquinolyl and phenyl, wherein n is preferably 0,1 or 2.

Where R₃ or R₄ is phenyl, said phenyl is preferably mono-di-or tri-substituted with one or more functional groups selected from halogen, C₁₋₄alkyl, C₁₋₄alkyloxy, C₁₋₄ alkylcarbonyl, nitro, especially preferred are methyl, methoxy, chloro and acetyl. Preferably, said phenyl is at least meta-substituted. Most preferred are mono-substituted phenyls, said substitution being at the meta position.

Preferably, one of R₃ and R₄ is hydrogen, methyl, ethyl, isopropyl, propyl and the other of R₃ and R₄ is substituted or unsubstituted phenyl or cyclohexyl.

Most preferably A is a substituted aniline.

It will be appreciated that formula (I) is intended to embrace all possible isomers, including optical isomers and mixtures thereof, including racemates.

The present invention includes within its scope prodrugs of the compounds of formula (I) above. In general, such prodrugs will be functional derivatives of the compounds of formula (I) which are readily convertible in vivo into the required compound of formula (I). Conventional procedures for the selection and preparation of suitable prodrug derivatives are described, for example, in “Design of Prodrugs”, ed H. Bungaard, Elsevier, 1985.

The scope of the invention also extends to salts, particularly physiologically acceptable salts and hydrates of the compounds of formula (I).

The pharmaceutically acceptable salts of the compounds of formula (I) include the conventional non-toxic salts or the quarternary ammonium salts of the compounds of formula (I) formed, eg, from non-toxic inorganic or organic acids. For example, such conventional non-toxic salts include those derived from inorganic acids, such as hydrochloric, hydrobromic, sulphuric, sulphamic, phosphoric, nitric and the like; and those prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulphanilic, 2-acetoxybenzoic, fumaric, toluenessulphonic, methanesulphonic, ethane disulphonic, oxalic, and the like.

The pharmaceutically acceptable salts of formula (I) also include those formed from a base, such as an alkali or alkaline earth metal hydroxide eg sodium, potassium, lithium, calcium or magnesium hydroxide, or an organic base, such as an amine eg dibenzylethylenediamine, trimethylamine, piperidine, pyrrolidine, benzylamine and the like, or a quarternary ammonium hydroxide eg tetramethylammonium hydroxide and the like.

The pharmaceutically acceptable salts of the present invention can be synthesised from any compound of formula (I) that contains a basic or acidic moiety by conventional chemical methods. Generally, the salts are prepared by reacting the free base or acid with a stoichiometric amount or with an excess of the desired salt-forming inorganic or organic acid or base in a suitable solvent.

According to a second aspect of the present invention, there is provided a method of producing a compound of Formula (I), comprising the steps of:

(i) providing a leaving group L at the C-3 position of compound (II) in which B is hydrogen or hydroxyl to give compound (III),

(ii) displacing said leaving group with an amino moiety A to give compound (I), wherein R₁, R₂, X₁ and X₂ are as defined above and A is selected from a nitrogen-containing heterocycle linked to the diazepine moiety via nitrogen and

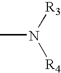

where R₃ and R₄ are as defined above.

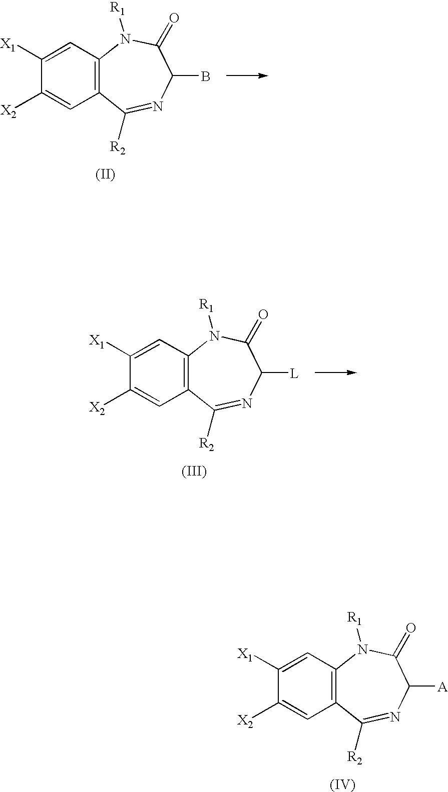

Leaving group L is conveniently halogen, preferably chloro, bromo or iodo.

When B is H, step (i) is conveniently achieved by free radical substitution, for example using N-bromosuccinimide (L=Br), or N-chlorosuccinimide (L=Cl).

When B is OH, step (i) is conveniently achieved by nucleophilic substitution, for example using thionyl chloride (L=Cl).

Step (i) may be a two step procedure. For example B═OH may be replaced by Cl using thionyl chloride in a first step and subsequently replaced by I in a nucleophilic substitution reaction using NaI in a polar solvent such as acetonitrile; I served as the leaving group in step (ii)

Step (ii) is readily achieved by displacing the leaving group (L) with an appropriate primary or secondary amine.

Where R₁ is required to be other than hydrogen, the method may include an initial step of alkylating a compound of formula (II) in which R₁ is hydrogen. Alternatively, said alkylation may be carried out between steps (i) and (ii) or after step (ii).

Alkylation may be carried out by standard methods, such as by reaction with an alkylating agent, for example the, corresponding halide (especially the chloride or bromide). Preferred alkylating agents corresponding to preferred substituents for R₁ include benzyl chloride, trimethylacetyl chloride, propargyl bromide, allyl bromide, ethyl chloroformate, phenacyl chloride or morpholinyl chloride.

The method may include a step, preferably a final step, of separating optical isomers. Such separation may be by any known means such as chiral HPLC of the enantiomeric forms, or classical resolution of the salts of tartaric acid. The method of choice is the formation of diastereoisomeric salts with L-tartaric acid, followed by recrystallisation of the SR and SS salts of the benzodiazepines. Alternatively L-lactic acid may be used for the separation of the recemic mixture.

The present invention also resides in the use of a compound of the first aspect as a CCK receptor ligand and/or as a CCK antagonist. Preferably, said use is as a selective CCK1 or CCK2 ligand.

The ability of the compounds of formula (I) to antagonise CCK by acting as CCK-receptor ligands makes these compounds useful as pharmacological agents for mammals, especially humans, for the treatment and prevention of disorders wherein CCK and/or gastrin may be involved.

Therefore the present invention in a third aspect resides in a method of treatment of a mammal afflicted with a CCK-related condition, or prophylaxis in a mammal at risk of a CCK-related condition by administration of a therapeutically effective amount of a compound of the first aspect of the invention.

The invention also resides in a pharmaceutical formulation comprising a compound of said first aspect in admixture with a pharmaceutically acceptable carrier therefor.

The invention further resides in the use of a compound of the first aspect in the preparation of a medicament, particularly a medicament for the treatment or prophylaxis of a CCK-related disorder.

Examples of CCK-related conditions states include GI disorders, especially such as irritable bowel syndrome, gastro-oesophageal reflux disease or ulcers, excess pancreatic or gastric secretion, acute pancreitis, or motility disorders; CNS disorders caused by CCK interactions with dopamine, such as neuroleptic disorders, tardive dyskinesia, Parkinson's disease, psychosis or Gilles de la Tourette syndrome; disorders of appetite regulatory systems; Zollinger-Ellison syndrome; antral G cell hyperplasia; or pain (potentiation of opiate analgesia).

The treatment of opiate-resistant severe clinical pain may represent the most, important of the CNS applications, but other applications based on the interaction between CCK and dopamine in forebrain could also deserve clinical exploration

The compounds of the invention may further be useful in the treatment or prevention of additional central nervous system disorders including neurological and psychiatric disorders. Examples of such central nervous system disorders include anxiety disorders and panic disorders, wherein CCK is involved. Additional examples of central nervous system disorders include panic syndrome, anticipatory anxiety, phobic anxiety, panic anxiety, chronic anxiety and endogeneous anxiety.

The compounds of of the invention may further be useful in the treatment of oncologic disorders wherein CCK may be involved. Examples of such oncologic disorders include small cell adenocarcinomas and primary tumours of the central nervous system glial and neuronal cells. Example of such adenocarcinomas and tumours include, but are not limited to, tumours of the lower oesophagus, stomach, intestine, colon and lung, including small cell lung carcinoma.

The compounds of the invention may further be used to control pupil constriction in the eye. The compounds may be used for therapeutic purposes during eye examinations and intra-ocular surgery in order to prevent miosis. They may further be used to inhibit miosis occurring in association with iritis, uveitis and trauma.

The compounds of the invention may further be useful for preventing or treating the withdrawal response produced by chronic treatment or abuse of drugs or alcohol. Such drugs include, but are not limited to, cocaine, alcohol or nicotine.

The compounds of the invention may also be useful as neuroprotective agents, for example, in the treatment and/or prevention of neuro-degenerative disorders arising as consequence of such pathological conditions as stroke, hypoglycaemia, cerebral palsy, transient cerebral ischaemic attack, cerebral ischaemia during cardiac pulmonary surgery or cardiac arrest, perinatal asphyxia, epilepsy, Huntingdon's chorea, Alzheimer's disease, amyotrophic lateral sclerosis, Parkinson's disease, olivo-pontocerebellar atrophy, anoxia such as from drowning, spinal cord and head injury, and poisoning by neurotoxins, including environmental neurotoxins.

The dosage administered to a patient will normally be determined by the prescribing physician and will generally vary according to the age, weight and response of the individual patient, as well as the severity of the patient's symptoms. However, in most instances, an effective therapeutic daily dosage will be in the range of from about 0.05 mg/kg to about 50 mg/kg of body weight and, preferably, of from 0.5 mg/kg to about 20 mg/kg of body weight administered in single or divided doses. In some cases, however, it may be necessary to use dosages outside these limits.

In the treatment of irritable bowel syndrome, for instance, 0.1 to 10 mg/kg of a CCK antagonist might be administered orally (p.o.), divided into two doses per day (b.i.d.). In treating delayed gastric emptying, the dosage range would probably be the same, although the drug might be administered either intravenously (i.v.) or orally, with the i.v. dose probably tending to be slightly lower due to a better availability. Acute pancreitis might be treated preferentially in an i.v. form, whereas spasm and/or reflex oesophageal, chronic pancreitis, post-vagotomy diarrhoea, anorexia or pain associated with biliary dyskinesia might indicate a p.o. form of administration.

In the effective treatment of panic syndrome, panic disorder, anxiety disorder and the like, preferably about 0.05 mg/kg to about 1.0 mg/kg of CCK antagonist may be administered orally (p.o.), in single or divided doses per day (b.i.d.). Other routes of administration are also suitable.

For directly inducing analgesia, anaesthesia or loss of pain sensation, the effective dosage range is preferably from about 100 mg/kg to about 1 mg/kg by intraperitoneal administration. Oral administration is an alternative route, as well as others.

While it is possible for an active ingredient to be administered alone as the raw chemical, it is preferable to present it as a pharmaceutical formulation. The formulations, both for veterinary and for human medical use, of the present invention comprise an active ingredient in association with a pharmaceutically acceptable carrier therefor and optionally other therapeutic ingredient(s). The carrier(s) must be ‘acceptable’ in the sense of being compatible with the other ingredients of the formulation and not deleterious to the recipient thereof.

Conveniently, unit doses of a formulation contain between 0.1 mg and 1 g of the active ingredient. Preferably, the formulation is suitable for administration from one to six, such as two to four, times per day. For topical administration, the active ingredient preferably comprises from 1% to 2% by weight of the formulation but the active ingredient may comprise as much as 10% w/w. Formulations suitable for nasal or buccal administration, such as the self-propelling powder-dispensing formulations described hereinafter, may comprise 0.1 to 20% w/w, for example about 2% w/w of active ingredient.

The formulations include those in a form suitable for oral, ophthalmic, rectal, parenteral (including subcutaneous, vaginal, intraperitoneal, intramuscular and intravenous), intra-articular, topical, nasal or buccal administration.

Formulations of the present invention suitable for oral administration may be in the form of discrete units such as capsules, cachets, tablets or lozenges, each containing a predetermined amount of the active ingredient; in the form of a powder or granules; in the form of a solution or a suspension in an aqueous liquid or non-aqueous liquid; or in the form of an oil-in-water emulsion or a water-in-oil emulsion. The active ingredient may also be in the form of a bolus, electuary or paste. For such formulations, a range of dilutions of the active ingredient in the vehicle is suitable, such as from 1% to 99%, preferably 5% to 50% and more preferably 10% to 25% dilution. Depending upon the level of dilution, the formulation will be either a liquid at room temperature (in the region of about 20° C.) or a low-melting solid.

Formulations for rectal administration may be in the form of a suppository incorporating the active ingredient and a carrier such as cocoa butter, or in the form of an enema.

Formulations suitable for parenteral administration comprise a solution, suspension or emulsion, as described above, conveniently a sterile aqueous preparation of the active ingredient that is preferably isotonic with the blood of the recipient.

Formulations suitable for intra-articular administration may be in the form of a sterile aqueous preparation of the active ingredient, which may be in a microcrystalline form, for example, in the form of an aqueous microcrystalline suspension or as a micellar dispersion or suspension. Liposomal formulations or biodegradable polymer systems may also be used to present the active ingredient particularly for both intra-articular and ophthalmic administration.

Formulations suitable for topical administration include liquid or semi-liquid preparations such as liniments, lotions or applications; oil-in-water or water-in-oil emulsions such as creams, ointments or pastes; or solutions or suspensions such as drops. For example, for ophthalmic administration, the active ingredient may be presented in the form of aqueous eye drops, as for example, a 0.1-1.0% solution.

Drops according to the present invention may comprise sterile aqueous or oily solutions. Preservatives, bactericidal and fungicidal agents suitable for inclusion in the drops are phenylmercuric salts (0.002%), be nzalkonium chloride (0.01%) and chlorhexidine acetate (0.01%). Suitable solvents for the preparation of an oily solution include glycerol, diluted alcohol and propylene glycol.

Lotions according to the present invention include those suitable for application to the eye. An eye lotion may comprise a sterile aqueous solution optionally containing a bactericide or preservative prepared by methods similar to those for the preparation of drops. Lotions or liniments for application to the skin may also include an agent t6 hasten drying and to cool the skin, such as an alcohol, or a softener or moisturiser such as glycerol or an oil such as castor oil or arachis oil.

Creams, ointments or pastes according to the present invention are semi-solid formulations of the active ingredient in a base for external application. The base may comprise one or more of a hard, soft or liquid paraffin, glycerol, beeswax, a metallic soap; a mucilage; an oil such as a vegetable oil, eg almond, corn, arachis, castor or olive oil; wool fat or its derivatives; or a fatty acid ester of a fatty acid together with an alcohol such as propylene glycol or macrogols. The formulation may also comprise a suitable surface-active agent, such as an anionic, cationic or non-ionic surfactant such as a glycol or polyoxyethylene derivatives thereof. Suspending agents such as natural gums may be incorporated, optionally with other inorganic materials, such as silicaceous silicas, and other ingredients such as lanolin.

Formulations suitable for administration to the nose or buccal cavity include those suitable for inhalation or insufflation, and include powder, self-propelling and spray formulations such as aerosols and atomisers. The formulations, when dispersed, preferably have a particle size in the range of 10 to 200μ.

Such formulations may be in the form of a finely comminuted powder for pulmonary administration from a powder inhalation device or self-propelling powder-dispensing formulations, where the active ingredient, as a finely comminuted powder, may comprise up to 99.9% w/w of the formulation.

Self-propelling powder-dispensing formulations preferably comprise dispersed particles of solid active ingredient, and a liquid propellant having a boiling point of below 18° C. at atmospheric pressure. Generally, the propellant constitutes 50 to 99.9% w/w of the formulation whilst the active ingredient constitutes 0.1 to 20% w/w. for example, about 2% w/w, of the formulation.

The pharmaceutically acceptable carrier in such self-propelling formulations may include other constituents in addition to the propellant, in particular a surfactant or a solid diluent or both. Surfactants are desirable since they prevent agglomeration of the particles of active ingredient and maintain the active ingredient in suspension. Especially valuable are liquid non-ionic surfactants and solid anionic surfactants or mixtures thereof. Suitable liquid non-ionic surfactants are those having a hydrophile-lipophile balance (HLB, see Journal of the Society of Cosmetic Chemists Vol. 1 pp. 311-326 (1949)) of below 10, in particular esters and partial esters of fatty acids with aliphatic polyhydric alcohols. The liquid non-ionic surfactant may constitute from 0.01 up to 20% w/w of the formulation, though preferably it constitutes below 1% w/w of the formulation. Suitable solid anionic surfactants include alkali metal, ammonium and amine salts of dialkyl sulphosuccinate and alkyl benzene sulphonic acid. The solid anionic surfactants may constitute from 0.01 up to 20% w/w of the formulation, though preferably below 1% w/w of the composition. Solid diluents may be advantageously incorporated in such self-propelling formulations where the density of the active ingredient differs substantially from the density of the propellant; also, they help to maintain the active ingredient in suspension. The solid diluent is in the form of a fine powder, preferably having a particle size of the same order as that of the particles of the active ingredient. Suitable solid diluents include sodium chloride, sodium sulphate and sugars.

Formulations of the present invention may also be in the form of a self-propelling formulation wherein the active ingredient is present in solution. Such self-propelling formulations may comprise the active ingredient, propellant and co-solvent, and advantageously an antioxidant stabiliser. Suitable co-solvents are lower alkyl alcohols and mixtures thereof. The co-solvent may constitute 5 to 40% w/w of the formulation, though preferably less than 20% w/w of the formulation. Antioxidant stabilisers may be incorporated in such solution-formulations to inhibit deterioration of the active ingredient and are conveniently alkali metal ascorbates or bisulphites. They are preferably present in an amount of up to 0.25% w/w of the formulation.

Formulations of the present invention may also be in the form of an aqueous or dilute alcoholic solution, optionally a sterile solution, of the active ingredient for use in a nebuliser or atomiser, wherein an accelerated air stream is used to produce a fine mist consisting of small droplets of the solution. Such formulations usually contain a flavouring agent such as saccharin sodium and a volatile oil. A buffering agent such as sodium metabisulphite and a surface-active agent may also be included in such a formulation which should also contain a preservative such as methylhydroxybenzoate.

Other formulations suitable for nasal administration include a powder, having a particle size of 20 to 500 microns, which is administered in the manner in which snuff is taken, ie by rapid inhalation through the nasal passage from a container of the powder held close up to the nose.

In addition to the aforementioned ingredients, the formulations of this invention may include one or more additional ingredients such as diluents, buffers, flavouring agents, binders, surface active agents, thickeners, lubricants, preservatives eg methylhydroxybenzoate (including anti-oxidants), emulsifying agents and the like. A particularly preferred carrier or diluent for use in the formulations of this invention is a lower alkyl ester of a C₁₈ to C₂₄ mono-unsaturated fatty acid, such as oleic acid, for example ethyl oleate. Other suitable carriers or diluents include capric or caprylic esters or triglycerides, or mixtures thereof, such as those caprylic/capric triglycerides sold under the trade name Miglyol, eg Miglyol 810.

Because these compounds antagonise the function of CCK in animals, they may also be used as feed additives to increase the food intake of animals, such as in a daily dosage of from about 0.05 to 50 mg/kg of body weight.

The present invention will now be illustrated by the following Examples.

### EXAMPLES

General Synthetic Methods

The majority of chemicals used were obtained from the laboratory and chemical stores. The remainder were ordered from Aldrich Catalogue Handbook of Fine Chemicals and Lancaster 1999/2000/2001.

Mass spectrometric analyses was obtained by Atmospheric Pressure Chemical Ionisation (APCI), negative or positive mode, using a Hewlett-Packard 5989b quadrupole instrument. This was connected to an electrospray 59987A unit with automatic injection (Hewlett-Packard 1100 series autosampler). Samples were dissolved in HPLC grade methanol, toluene or acetonitrile. Both Proton and Carbon NMR spectra were obtained on a brucker AC 250 instrument, operating at 250 MHz, calibrated with the solvent reference peak or TMS.

IR spectra were plotted from KBr discs on a Mattson 300 FTIR Spectrophotometer. Melting points were recorded from a Stuart Scientific Melting Point (SMP1) and are uncorrected. Analytical Thin Layer Chromatography was obtained using aluminium sheets, silica gel₆₀ F254 and visualized using ultraviolet light. Preparative chromatography was performed on 20×20 cm silica gel TLC plates from Aldrich. Jencons sonomatic sonicator (SO175) was used to prepare samples for screening. All compounds for screening were dissolved in HPLC grade DMSO.

Small scale solution syntheses was carried out on a carousel reaction stations (RR 98030), with 12 place carousel reaction station and reflux head and 12× flexible tubing from Radleys, on a RCT basic hotplate from IKA Labortechnik with IKATRON ETS D3 temperature controller or by using heating blocks (TECHNE Dri-block-DB-3A).

Pharmacological Methods: ^{[125]}I-CCK-8 Receptor Binding Assay:

CCK_{A} and CCK_{B} receptor binding assays were performed, by using guinea pig cerebral cortex (CCK_{B}) or rat pancreas (CCK_{A}). Male guinea pig brain tissues were prepared according to the modified method described by Saita et al [(1994), Characterization of YM022: its CCKB/gastrin receptor binding profile and antagonism to CCK-8-induced Ca2+ mobilization., Eur. J. Pharmacol., 269, 249-254]. Pancreatic membranes were prepared in a similar way but by Charpentier et al [(1988), Cyclic cholecystokinin analogues with high selectivity for central receptors., Proc Natl Acad Sci USA, 85, 1968-1972]. The in vivo CCK binding assay: Tissues were homogenised in ice cold sucrose (0.32 M, 25 ml) for 15 strokes at 500 rpm and centrifuged at 13000 rpm for 10 mins. The supernatant was re-centrifuged at 13000 rpm for 20 mins. The resulting pellet was re-dispersed to the required volume of buffer at 500 rpm and stored in aliquots at 70° C.

Binding was achieved using a radioligand ¹²⁵I-Bolton-Hunter labeled CCK, NEN at 25 pM. The samples were incubated {with membranes (0.1 mg/ml)} in 20 mM Hepes, 1 mM EGTA, 5 mM MgCl₂, 150 mm NaCl, 0.25 mg/ml bacitracin at pH 6.5 for 2 hrs at RT and then suspended by centrifugation at 1100 rpm for 5 minutes. The membrane pellets were washed twice with water and the bound radioactivity was measured in a Packard Cobra Auto-gamma counter (B5005). All binding assays were carried out with L-363, 260 as an internal non-specific standard. Controls (no compound) were also added. All samples were made in duplicate and repeated twice. All compounds were initially screened for percentage inhibition at 20 μM. Samples showing an average inhibition of <35% were diluted to 2 μM and re-screened and if active diluted again. This enabled the calculation of IC₅₀'s of the most active compounds.

Preparation of Starting Materials

Description 1: Preparation of Oxazepam Starting Material (4) (Scheme 1)

The addition of hydroxylamine to 2 amino-5-chlorobenzophenone (1) gave the imine (2) product (89%). Reacting this (2) under cooling conditions gave 2-chloroacetamido-5-chlorobenzophenone (3) (96%). Keeping the solution basic, to neutralise the by-product HCl, on stirring overnight resulted in the formation of oxazepam salt, which was acidified to give oxazepam (60%) (4) and the undissolved salt (22%).

Description 2: Preparation of N-Alkylated Oxazepam Starting Material (Scheme 1)

Alkylation (substituent R) was achieved by reacting oxazepam (4) with a 50% suspension of NaH, in dry dimethyl formamide (DMF). After stirring at room temperature, the appropriate alkylating agent was added dropwise and left for 45 minutes. Work-up was accomplished with ethylacetate and then washing with water and brine. Column chromatography (ether/petrol ether 1:2) yielded the pure product (8).

Description 3: Preparation of Diazepam Starting Material (Scheme 1)

Diazepam (10) was synthesised according to the standard literature procedure. Briefly, the ketone building block (1) was acetylated with chloroacetylchloride in anhydrous ether at 0° C. to give (9) which was not isolated. (9) was then refluxed with urotropin (hexamethylenetetramine) for 16 hours to enable cyclisation (the Delepine reaction) to give the amino-aceto-amide compound, which was not isolated. The whole mixture was cooled with diazepam crystals (10) precipitating out.

### Description A: Preparation of N-(2-Benzoyl-4-chlorophenyl)-2-chloroacetamide (9)

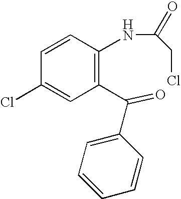

A solution of 2-amino-5-chlorobenzophenone (1) (11.6 g, 50 mmol) in anhydrous ether (75 ml) was stirred and cooled in an ice bath to 0-5° C. Chloroacetyl chloride (55 mmol, 4.4 ml) in ether (25 ml) was added dropwise. Precipitation of the title compound (9) occurred. The suspension was stirred for half an hour at 0-5° C. and for 2 hours at room temperature. The solid product was collected by filtration and crystallised with toluene.

Yield: 91%.

R_{f} (ether)=079

Mol. Weight: 308.1.

Mol. Formula: C₁₅H₁₁Cl₂N₂O.

MS (APCI (+)) m/s: 308 M+H, 231 M+m/z.

¹H-NMR (CDCl₃) 300K δ 4.2 (s, 2H, NHCOCH₂Cl), 7.3-7.8 (m, Ar-8H), 11.5 (s, 1H, NHCOCH₂Cl) p.p.m.

### Description B: Preparation of 7-Chloro-5-phenyl-1,3-dihydro-2H-1,4-benzodiazepin-2-one (Diazepam 10)

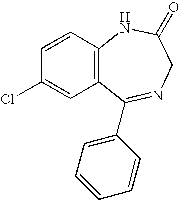

A mixture of the precursor 2-chloro-N-{4-chloro-2-[(hydroxyimino)(phenyl)methyl]phenyl}acetamide (9) (13 g, 42 mmol, 11.9 g of urotropine (85 mmol), HCl, (20 ml 2N aqueous), methanol (80 ml) and water (10 ml) were added (pH of solvent mixture was≅5) and refluxed for 16 hours. The mixture was cooled in an ice bath and the precipitated crystals were filtered. The crystals were washed with a 10 ml ice-cold mixture of methanol/water (1:1). The product (10) was dried at 60° C. under reduced pressure overnight.

Yield: 82%.

R_{f} (ether)=0.42

Mol. Weight: 270.2.

Mol. Formula: C₁₅H₁₁ClN₂O.

MS (APCI(+)): 271, 272 (M+1) m/z.

IR (KBr-disc) υ max: 3420, 3312, 3207, 2960, 1679, 1534, 1213 & 794 cm⁻¹.

¹H NMR (DMSO-d₆) 300K δ: 4.38 (s, 2 H, C₃), 7.65 (m, Ar-8H), 10.0 (s, NH) p.p.m.

¹³C NMR (DMSO-d₆) 300K δ: 55.2 (C3), 121.9, 128.6, 129.5, 129.7, 130.8, 131.4, 137.3, 139.4(Ar—C), 164.8(C═N), 168.7 (C═O) p.p.m.

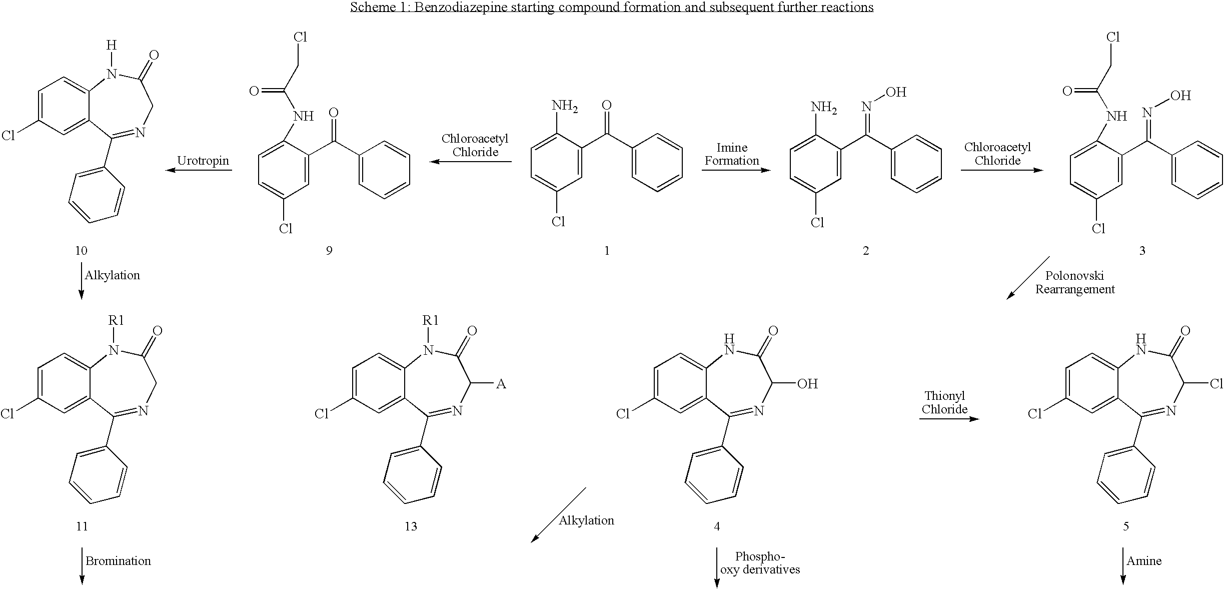

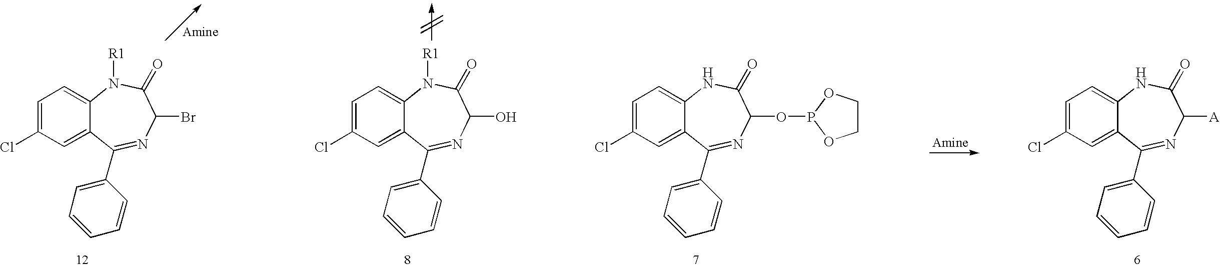

Synthesis of Initial 3-Amino-substituted Benzodiazepines from Oxazepam Route A: Oxazepam (4) (0.1 g, 3.5×10⁻⁴ mol) was treated with thionyl chloride (4 Eq, 0.1 ml) and heated to 60° C. for 1.5 hours. The resulting intermediate (5), a yellow solid, was washed with dry diethyl ether (twice) to remove any excess thionyl chloride. The appropriate amine (2.5 Eq, 1.1×10⁻³ mol), with TEA (drops) was added with dry DCM (15 ml) to maintain the solution basic, and refluxed for two to three hours. The organic phase was washed with hydrochloric acid (pH 4.0-5.0) and optionally with water to remove any unreacted amine, and dried over sodium sulphate. Excess hexane was added and the mixture was allowed to stand overnight. The precipitate was filtered, washed with hexane and dried.

Route B: Oxazepam (4) (0.2 g, 6.8×10⁻⁴ mol) in dry THF (13 ml), and sodium hydride (to remove the hydroxyl proton) (60% in mineral oil, 0.052 g, 1.0×10⁻³ mol) was stirred for 1 hour at room temperature under argon. The solution turned light brown in colouration. After 1 hour 2-chloro-1,3,2-dioxaphospholane (1.0×10⁻³ mol) was added drop-wise and stirred at room temperature for 2.5 hours (7). The appropriate amine (1.8×10⁻³ mol) was added and left overnight at room temperature under argon.

TLC suggested formation of product was optimal, when left overnight. The filtrate was purified by flash chromatography, using ethyl acetate as the mobile phase The synthetic route B was investigated to compare yields and reliability with route A. Yields of formation were generally higher with the precipitation method B but the reagents are generally more expensive and A is ideal for the large scale production.

Synthesis of 3-Amino-substituted Benzodiazepines from Diazepam

When R was other than H, the diazepam was alkylated, using the standard conditions of Description 2. The bromination procedure involved the use of NBS, CCl₄ and a halocarboxylic acid (TFA, trifluoroacetic acid) in a radical reaction. The reaction was initially stirred at room temperature, then refluxed vigorously for 1-1.5 hours. The residue was decanted, washed and dried to give an unstable yellowy brown oil, in a high yield (91%) (11). The required amine (2.5 equivalents) was added in dry dichloromethane, with drops of TEA and left at 40° C. overnight. The mixture was washed with water and dried, with the DCM removed in vacuo. After column chromatography on the mixture (ether:petroleum ether 1:2), the product (yellow powder) was isolated (12).

### Examples 1-11

### Biological Results—Table 1: Structure and Activity of the Initially Synthesised Analogues

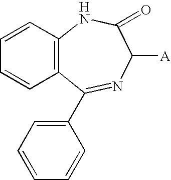 General structure of the analogues

| | | MS | CCK_{B} |
|---|---|---|---|
| Exam- | | (M + H) | IC₅₀ |
| ple | A | [mz] | [μM] |
| 1 | 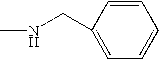 | 376 | 1.2 |
| 2 | 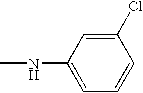 | 396 | 0.32 |
| 3 | 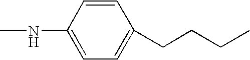 | 418 | 0.58 |
| 4 | 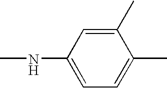 | 390 | 0.66 |
| 5 | 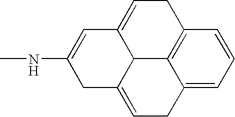 | 486 | 0.84 |
| 6 | 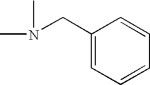 | 390 | 0.86 |
| 7 | 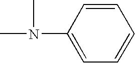 | 390 | 0.31 |
| 8 | 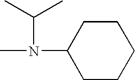 | 410 | 0.19 |
| 9 | 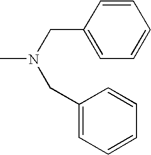 | 466 | 0.26 |
| 10 | 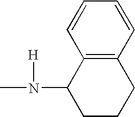 | 402 | 0.42 |
| 11 | 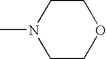 | 356 | 2.7 |

Biological evaluation in the radiolabeled receptor binding assay showed good CCK_{B} binding affinities (IC₅₀=in the nanomolar range). This initial screening result suggests that anilines, particularly secondary, demonstrate the highest CCK_{B} binding activity.

The most active compound in this series was Example 8. It comprises cyclohexylamine having an isopropyl substituent (IC₅₀=190 nM). From the initial screening results, aniline analogues and cyclohexylamine derivatives showed the best in vitro activity

### Examples 12-34

### Synthesis and Biology of Further 3-Amino-Substituted Benzodiazepines

Further compounds and other 3-amino substituted 1,4-benzodiazepine-2-one derivatives were synthesised, following the procedure of either route A, the thionyl chloride method or route B, using a phospho-oxy derivative. When route A was used, after the work-up with water, the compounds were isolated by precipitation with hexane. This was achieved by allowing the solution to stand overnight, with an excess of hexane and then washing and drying the product.

### Example 12

### Preparation of 7-chloro-3-(3,5-dimethyl-1H-pyrazol-1-yl)-5-phenyl-1,3-dihydro-2H-1,4-benzodiazepin-2-one

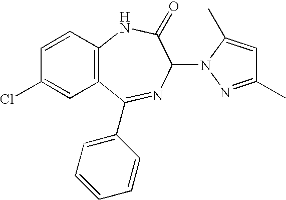

Following synthetic route A, the title compound was prepared and identified, as follows:

Yield: 69.0%.

Mol. Formula: C₂₀H₁₇ClN₄O.

Mol. Weight: 364.8.

IR (KBr-disc) υ max: 3400, 3020, 2930, 2970, 1695, 1320, 1215, 1100, 790 cm⁻¹.

MS (APCI(+)): 365, 367 (M+1), 269, 271 (M+) m/z.

¹H NMR (DMSO-d₆) 300K δ: 11.13 (s, NH), 7.73 (dd, Ar—H, J=8.8 Hz), 7.46-7.56 (m, phenyl-5H), 7.38 (d, Ar—H, J=8.8 Hz), 7.32 (s, Ar—H), 5.71 (s, Ar—H), 4.79 (s, C3-H), 2.36 (s, —OCH₃), 2.09 (s, —OCH₃) p.p.m.

¹³C NMR (DMSO-d₆) 300K δ: 11.0 (CH₃), 13.7 (CH₃), 71.6 (C3), 106.3, 122.9, 125.5 (2xC), 128.2, 129.3, 130.3 (2xC), 130.8, 131.2, 134.1, 135.0, 139.2, 140.7, 156.1, (Ar—C), 162.2 (C═O), 170.9 (C═N) p.p.m.

### Example 13

### Preparation of 7-chloro-3-(4-amino-1,5-dimethyl-2-phenyl-1,2-dihydro-3H-pyrazol-3-one -5-phenyl-1,3-dihydro-2-H-1,4-benzodiazepine-2-one

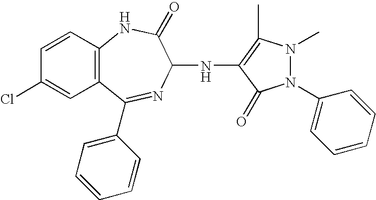

Following synthetic route A, the title compound was prepared and identified, as follows:

Yield: 58%.

Mol. Formula: C₂₆H₂₂ClN₅O₂.

Mol. Weight: 471.94.

IR (KBr-disc) υ max: 3448, 3254, 2927, 1716, 1625, 1448, 1313, 1168, 696 cm⁻¹.

MS (APCI(+)): 472, 474 (M+1), 454, 456 (M+) m/z.

¹H NMR (DMSO-d₆) 300K δ: 10.95 (s, NH), 7.66-7.70 (dd, Ar—H, J=9.0 Hz), 7.22-7.53 (m, Phenyl-13H), 5.08 (s, C₃—H), 2.86 (s, N—CH₃), 2.20 (s, C—CH₃) p.p.m.

¹³C NMR (DMSO-d₆) 300K δ: 11.0 (C—CH₃), 38.2 (N—CH₃), 70.1 (C₃), 118.4 (2xC), 122.9 (2xC), 123.9, 126.1, 127.2, 128.3, 129.0, 129.5 (2xC), 129.8, 129.9, 131.1, 132.5, 135.8 (2xC), 138.2, 138.6, 143.0 (Ar—C), 162.0 (C═O), 164.9 (NH—C═O), 169.0 (C═N) p.p.m.

### Example 14

### Preparation of 3-(1-phenylpiperazin-4-yl)-7-chloro-5-phenyl-1,3-dihydro-2-H-1,4-benzodiazepine-2-one

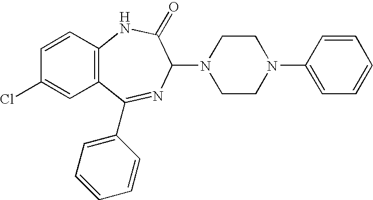

Following synthetic route B, the title compound was prepared and identified, as follows:

R_{f} (ethylacetate)=0.37.

Yield: 58%.

Mol. Formula: C₂₅H₂₃ClN₄O.

Mol. Weight: 430.94.

IR (KBr-disc) υ max: 3434, 3049, 2921, 2417, 1704, 1596, 1482, 1324, 1091, 685 cm⁻¹.

MS (APCI(+)): 431, 433 (M+1), 269, 271 (M+) m/z.

¹H NMR (DMSO-d₆) 300K δ: 11.79 (s, NH), 7.76-7.82 (dd, Ar—H, J=9.8 Hz), 7.49-7.65 (m, Phenyl-6H), 7.34 (s, Ar—H), 7.26-7.32 (d, Ar-2H, J=8.3 Hz), 7.02-7.05 (d, Ar-2H, J=8.1 Hz), 6.85-6.91 (t, Ar—H, J=7.3, 7.2 Hz), 5.30 (s, C₃—H), 3.40-3.53 (m, —CH₂— 8H) p.p.m.

¹³C NMR (DMSO-d₆) 300K δ: 58.5 (2x-CH₂—N—C₃), 70.0 (2x-CH₂—N—Ar), 71.8 (C₃), 116.4 (2xC), 120.5, 124.7, 128.1, 128.3, 129.2, 129.7 (2xC), 130.4 (2xC), 132.2, 133.3, 134.9, 137.6, 137.8 (2xC), 150.0 (Ar—C), 164.2 (C═O), 167.9 (C═N) p.p.m.

### Example 15

### Preparation of N-benzylpiperidin-4-amino-7-chloro-5-phenyl-1,3-dihydro-2H-1,4-benzodiazepin-2-one

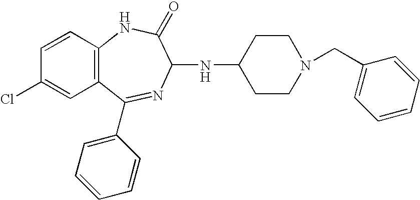

Following synthetic route B, the title compound was prepared and identified, as follows:

Yield: 35.0%

R_{f} (ethylacetate)=0.44.

Mol. Formula: C₂₇H₂₇ClN₄O.

Mol. Weight: 458.9.

IR (KBr-disc) υ max: 3434, 2828, 2358, 1994, 1602, 1481, 1318, 1120, 742, 699 cm⁻¹.

MS (APCI(+)): 459, 461 (M+1), 269, 271 (M+) m/z.

¹H NMR (DMSO-d₆) 300K δ: 11.59 (s, NH), 7.66-7.74 (m, Ar—H, 1.3H), 4.30 (s, C3-H), 2.74-3.04 (m, CH), 2.66-2.83 (m,—CH₂—Ar), 1.92-2.09 (m,—CH₂—, 4H), 1.40-1.55 (m,—CH₂—, 4H) p.p.m.

¹³C NMR (DMSO-d₆) 300K δ 32.0 (—CH₂-x2), 38.9 (—CH₂—N x2), 52.5 (CH), 73.1 (C3), 123.8, 127.3, 128.3 (2xC), 128.6, 128.7, 128.9 (2xC), 129.3 (2xC), 129.7 (2xC), 129.9, 131.0, 132.2, 138.3, 138.8, 139.1 (Ar—C), 164.9 (C═O), 167.2 (C═N) p.p.m.

### Example 16

### Preparation of 3-[8-aza-1,4-diaoxaspiro[4.5]decanyl]-7-chloro-5-phenyl-1,3-dihydro-2-H-1,4-benzodiazepine-2-one

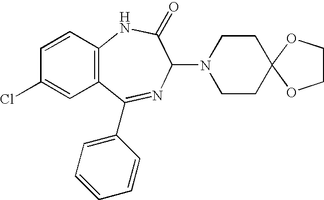

Following synthetic route A, the title compound was prepared and identified, as follows:

Yield: 62%.

Mol. Formula: C₂₂H₂₂ClN₃O₃.

Mol. Weight: 411.87.

IR (KBr-disc) υmax: 3436, 2915, 2473, 2358, 1706, 1606, 1478, 1081, 695 cm⁻¹.

APCI(+): 412, 414 (M+1), 269, 271 (M+) m/z.

¹H NMR (DMSO-d₆) 300K δ 11.65 (s, NH), 7.76-7.80 (dd, Ar—H, J=9.0 Hz), 7.49-7.69 (m, Phenyl-5H), 7.40-7.43 (d, Ar—H, J=9.0 Hz), 7.31 (s, Ar—H), 5.24 (s, C₃—H), 3.39-3.52 (m, —CH₂— 12H) p.p.m.

¹³C NMR (DMSO-d₆) 300K δ 58.6 (2x-CH₂—C), 70.1 (2x-CH₂—N), 71.8 (2x-CH₂—O), 77.8 (C₃), 104.7 (C—O), 124.7, 128.1 (2xC), 128.3, 129.2 (2xC), 130.4, 132.1, 133.2, 137.5, 137.7 (Ar—C), 164.5 (C═O), 167.8 (C═N) p.p.m.

### Example 17

### Preparation of 7-chloro-3-[3,4-dihydroquinolin-1(2H)-yl]-5-phenyl-1,3-dihydro-2H-1,4-benzodiazepin-2-one

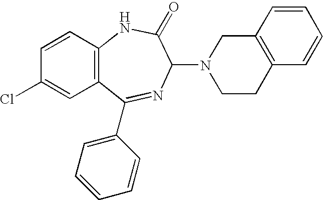

Following synthetic route A, the title compound was prepared and identified, as follows:

Yield: 66.0%.

Mol. Formula: C₂₄H₂₀ClN₃O.

Mol. Weight: 401.9.

IR (KBr-disc) υ max: 3440, 3050, 2930, 2850, 2360, 1700, 1610, 1480, 830, 740 cm⁻¹.

MS (APCI(+)): 402, 404 (M+1), 384, 386 (—H₂O), 269, 271(M+) m/z.

¹H NMR (DMSO-d₆) 300K δ: 10.91 (s, NH), 7.72 (dd, Ar—H, J=8.7 Hz), 7.47-7.55 (m, phenyl-5H), 7.37 (d, Ar—H, J=8.8 Hz), 7.28 (s, Ar—H), 6.95 (d, Ar—H, J=7.2 Hz), 6.86 (d, Ar—H, J=7.4, 7.6 Hz), 6.52 (t, Ar—H, J=7.4, 7.2 Hz), 6.22 (d, Ar—H, J=8.2 Hz), 5.22 (s, C3-H), 4.09 (m, —CH—), 3.62 (m, —CH—), 2.77 (m, —CH₂—), 1.98 (m, —CH₂—) p.p.m.

¹³C NMR (DMSO-d₆) 300K δ: 22.0, 28.5, 45.5 (—CH₂—), 78.5 (C3), 111.5, 117.5, 122.6, 122.7, 125.6 (2xC), 128.1, 128.2, 128.3, 129.3, 129.9, 130.0 (2xC), 130.5, 131.7, 136.9, 138.1, 148.1, (Ar—C), 165.1 (C═O), 166.3 (C═N) p.p.m.

### Example 18

### Preparation of 3-(2 acetylanilino)-7-chloro-5-phenyl-1,3-dihydro-2H-1,4-benzodiazepin-2-one

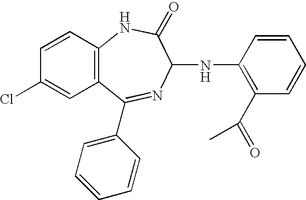

Following synthetic route B, the title compound was prepared and identified, as follows:

Yield: 43.0%.

R_{f} (ethylacetate)=0.35.

Mol. Formula: C₂₃H₁₈ClN₃O.

Mol. Weight: 403.8.

IR (KBr-disc) υ max: 3432, 2923, 2364, 1696, 1612, 1465, 1318, 1094, 742, 699 cm⁻¹.

MS (APCI(+)): 404,406 (M+1), 386, 388 (—H₂O), 269, 271 (M+) m/z.

¹H NMR (DMSO-d₆) 300K δ: 11.50 (s, NH), 7.74-7.68 (m, Ar—H, 12H), 4.30 (s, C3-H), 3.42 (s, CH₃) p.p.m.

¹³C NMR (DMSO-d₆) 300K δ: 33.8 (CH₃), 73.2 (C3), 123.8, 127.2, 127.3, 128.3, 128.6 (2xC), 128.7, 128.8, 128.9.(2xC), 129.3, 129.7, 129.9, 130.1, 132.2, 138.3, 138.9, 139.1, (Ar—C), 162.5, 164.7 (C═O), 169.2 (C═N) p.p.m.

### Example 19

### Preparation of 7-chloro-3-(3-methoxyanilino)-5-phenyl-1,3-dihydro-2H-1,4-benzodiazepin-2-one

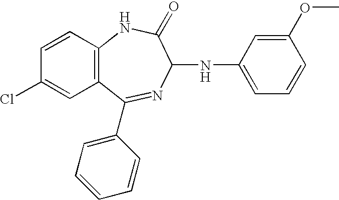

Following synthetic route A, the title compound was prepared and identified, as follows:

Yield: 45.0%.

Mol. Formula: C₂₂H₁₈ClN₃O₂.

Mol. Weight: 391.9.

IR (KBr-disc) υ max: 3445, 3210, 3080, 2940, 1690, 1520, 1495, 1230, 1140, 1030 cm⁻¹. MS (APCI(+)): 392, 394 (M+1), 374, 376 (−H₂O), 269, 271 (M+) m/z.

¹H NMR (DMSO-d₆) 300K δ: 11.20 (s, NH), 7.65 (dd, Ar—H, J=8.8 Hz), 7.43-7.51 (m, phenyl-5H), 7.31 (s, Ar—H), 7.30 (d, Ar—H, J=8.7 Hz), 6.98 (t, Ar—H, J=8.0, 8.0 Hz), 6.45 (d, Ar—H, J=7.5 Hz), 6.27 (s, Ar—H), 6.22 (m, Ar—H), 4.89 (d, C3-H, J=7.5 Hz), 3.66 (s, OCH₃) p.p.m.

¹³C NMR (DMSO-d₆) 300K δ: 55.7 (OCH₃), 66.9 (C3), 104.5, 107.9, 113.0, 122.7, 125.7 (2xC), 128.0, 128.5, 129.8 (2xC), 129.9, 130.8, 131.5, 137.1, 138.2, 142.1 (Ar—C), 160.1 (Ar—O) 164.1(C═O), 167.9 (C═N) p.p.m.

### Example 20

### Preparation of 7-chloro-3-(3,4-dimethoxyanilino)-5-phenyl-1,3-dihydro-2H-1,4-benzodiazepin-2-one

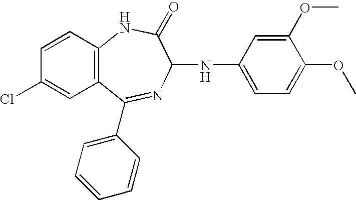

Following synthetic route A, the title compound was prepared and identified, as follows:

Yield: 50.0%.

Mol. Formula: C23H₂₀ClN₃O₃.

Mol. Weight: 421.9.

IR (KBr-disc) υ max: 3450, 3215, 3070, 2940, 1695, 1515, 1495, 1230, 700 cm⁻¹.

MS (APCI(+)): 422, 426 (M+1), 404, 406 (−H₂O), 269, 271 (M+) m/z.

¹H NMR (DMSO-d₆) 300K δ: 11.15 (s, NH), 7.70 (dd, Ar—H, J=8.7 Hz), 7.43-7.48 (m, phenyl-5H), 7.36 (s, Ar—H), 7.33 (d, Ar—H, J=8.8 Hz), 6.80 (d, Ar—H, J=8.7 Hz), 6.19 (dd, Ar—H, J=8.7 Hz), 6.06 (d, Ar—H, J=7.1 Hz), 5.97 (s, Ar—H), 5.03 (d, C3-H, J=7.1 Hz), 3.82 (s, OCH₃), 3.61(s, OCH₃) p.p.m.

¹³C NMR (DMSO-d₆) 300K δ: 55.1, 65.5 (OCH₃), 67.5 (C3), 105.1, 111.7, 122.9, 125.4 (2xC), 128.1, 128.6, 129.9 (2xC), 130.0, 130.8, 132.1, 136.5, 137.1, 140.2 (Ar—C), 152.7, 153.1 (Ar—O—), 165.2 (C═O), 165.9 (C═N) p.p.m

### Example 21

### Preparation of 3-(4-acetyl-3,5-dimethoxyanilino)-7-chloro-5-phenyl-1,3-dihydro-2H-1,4-benzodiazepin-2-one (compound 3.4.13)

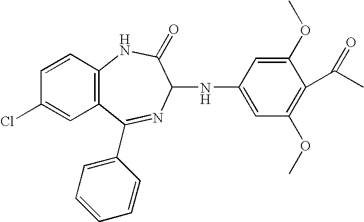

Following synthetic route B, the title compound was prepared and identified, as follows:

Yield: 70.0%.

R_{f} (ethylacetate)=0.35.

Mol. Formula: C₂₅H₂₂ClN₃O₄

Mol. Weight: 463.9.

IR (KBr) υ max: 3435, 3025, 2970, 2915, 1700, 1330, 1220, 7200 cm⁻¹.

MS (APCI(+)): 464, 466 (M+1), 269, 271(M+) m/z.

¹H NMR (CDCl₃) 300K δ: 12.85 (s, NH), 8.99 (d, NH, J=8.9 Hz), 7.80 (d, Ar—H, J=7.0 Hz), 7.53-7.67 (m, phenyl-5H), 7.27-7.53 (m, Ar-3H), 7.09 (s, Ar—H), 6.10 (s, C3-H), 4.08 (s, CH₃), 3.88 (s, OCH₃), 3.84 (s, OCH₃) p.p.m.

¹³C NMR (CDCl₃) 300K δ: 32.3 (CH₃), 55.7, 55.9 (OCH₃), 70.1 (C3), 93.8 (2xC), 107.6, 122.6, 125.8 (2xC), 128.1, 128.7, 129.5, 129.9 (2xC), 130.6, 132.6, 135.9, 136.4, 149.7, 165.1 (2xC), (Ar—C), 165.1 (C═O), 168.2, 168.9 (Ar—CO), 200.1 (C═N) p.p.m.

### Example 22

### Preparation of 3-anilino-7-chloro-5-phenyl-1,3-dihydro-2H-1,4-benzodiazepin-2-one

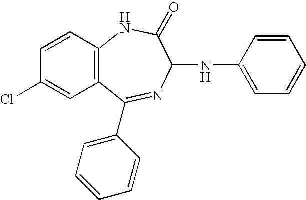

Following synthetic route A, the title compound was prepared and identified, as follows:

Yield: 80.0%.

Mol. Formula: C₂₁H₁₆ClN₃O.

Mol. Weight: 361.8.

IR (KBr-disc) υ max: 3425, 3025, 3065, 2930, 1710, 1590, 1440, 1340, 1100, 730, 700 cm⁻¹.

MS (APCI(+)): 362, 364 (M+1), 344, 346 (—H₂O), 269, 271 (M+) m/z.

¹H NMR (DMSO-d₆) 300K δ: 11.09 (s, NH), 7.70 (dd, Ar—H, J=8.7 Hz), 7.42-7.50 (m, phenyl-5H), 7.36 (d, Ar—H, J=8.8 Hz), 7.32 (s, Ar—H), 7.23 (t, Ar-2H, J=7.7, 7.8 Hz), 7.09 (t, Ar—H, J=7.6, 7.3 Hz), 6.96 (d, Ar-2H, J=7.7 Hz), 4.98 (s, C3-H) p.p.m.

¹³C NMR (DMSO-d₆) 300K δ: 67.4 (C3), 114.2 (2xC), 117.9, 122.8, 125.5 (2xC), 127.1, 128.8, 129.9 (2xC), 131.7, 136.9, 138.0, 150.2, (Ar—C), 165.1 (C═O), 167.9 (C═N) p.p.m.

### Example 23

### Preparation of 3-(benzylamino)-7-chloro-5-phenyl-1,3-dihydro-2H-1,4-benzodiazepin-2-one

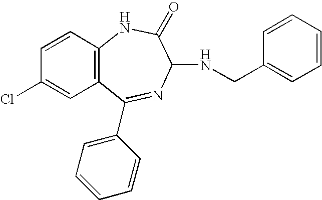

Following synthetic route A, the title compound was prepared and identified, as follows:

Yield: 62.0%;

Mol. Formula: C₂₂H₁₈ClN₃O.

Mol. Weight: 375.9.

IR (KBr-disc) υ max: 3430, 3335, 2975, 1705, 1580, 1330, 1095, 705 cm⁻¹.

MS (APCI(+)): 276, 278 (M+1), 358, 360 (−H₂O), 269, 270 (M+) m/z.

¹H NMR (DMSO-d₆) 300K δ: 11.57 (s, NH), 7.77 (dd, Ar—H, J=8.7 Hz), 7.53-7.62 (m, phenyl-5H), 7.53 (d, Ar—H, J=8.8 Hz), 7.40-7.44 (m, amine phenyl-5H), 7.30 (s, Ar—H), 5.75 (s, —CH₂—), 5.11 (s, C₃—H) p.p.m.

¹³C NMR (DMSO-d₆) 300K δ: 52.1 (CH₂), 74.2 (C3), 121.2, 125.8 (2xC), 126.5, 128.3, 128.5, 129.2 (2xC), 130.1 (2xC), 130.3, 130.6, 130.8 (2xC), 131.0, 134.2, 136.1, 136.9 (Ar—C), 162.1 (C═O), 167.1 (C═N) p.p.m.

### Example 24

### Preparation of 7-chloro-3-(methylanilino)-5-phenyl-1,3-dihydro-2H-1,4-benzodiazepin-2-one

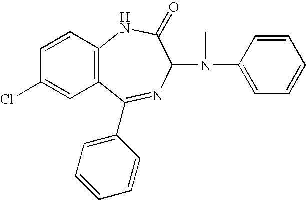

Following synthetic route A, the title compound was prepared and identified, as follows:

R_{f} (ether)=0.59.

Mol. Formula: C₂₂H₁₈ClN₃O.

Mol. Weight: 375.9.

IR (KBr-disc) υ max: 3430, 3216, 3129, 2923, 2851, 2358, 1708, 1596, 1496, 1318, 114, 693 cm⁻¹.

MS (APCI(+)): 376, 378 (M+1), 269, 271 (M+) m/z.

¹H NMR (DMSO-d₆) 300K δ: 3.40 (s, CH₃), 5.24 (s, C3-H), 6.68-6.71 (d, Ar-2H, J=8.8 Hz), 7.12-7.18 (t, Ar-2H, J=7.3, 8.5 Hz), 7.28 (s, Ar—H), 7.34-7.38 (d, Ar—H, J=8.8 Hz), 7.48-7.55 (m, phenyl-5H), 7.68-6.73 (dd, Ar—H, J=8.7 Hz), 11.89 (s, NH) p.p.m.

¹³C NMR (DMSO-d₆) 300K δ: 70.1, 70.8 (CH₃ isomers), 83.4 (C3), 117.8, 123.2, 123.3, 128.2 (2xC), 128.3, 128.9 (2xC), 129.4, 129.4 (2xC), 130.0, 130.3 (2xC), 131.7, 136.9, 138.1 (Ar—C), 165.1(C═O), 169.4 (C═N) p.p.m.

### Example 25

### Preparation of 7-chloro-3-(methylbenzyl)-5-phenyl-1,3-dihydro-2H-1,4-benzodiazepin-2-one

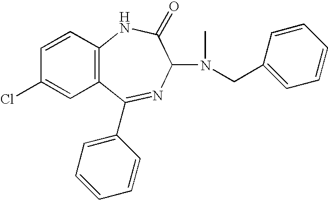

Following synthetic route A, the title compound was prepared and identified, as follows:

R_{f} (ether)=0.63.

Mol. Formula: C₂₃H₂₀ClN₃O.

Mol. Weight: 389.9.

IR (KBr-disc) υ max: 3398, 3324, 3128, 2899, 2832, 1767, 1522, 1477, 1320, 1150, 683 cm⁻¹.

MS (APCI(+)): 390, 391 (M+1), 269, 271 (M+) m/z.

¹H NMR (DMSO-d₆) 300K δ: 3.43 (s, CH₃), 4.21 (m, 2H, —CH₂—), 5.13 (s, C3-H), 6.65-6.70 (d, Ar-2H, J=8.8 Hz), 7.11-7.19 (t, Ar-2H, J=7.9, 8.5 Hz), 7.29 (s, Ar—H), 7.34-7.40 (d, Ar—H, J=8.9 Hz), 7.47-7.58 (m, phenyl-5H), 7.68-6.73 (dd, Ar—H, J=8.8 Hz), 11.59 (s, NH) p.p.m.

¹³C NMR (DMSO-d₆) 300K δ: 66.7 (—CH₂—), 70.4, 71.2 (CH₃ isomers), 84.4 (C3), 117.83, 123.0; 123.1, 128.2 (2xC), 128.5, 128.8.(2xC), 129.2, 129.3 (2xC), 130.2, 130.1 (2xC), 131.7, 136.8, 138.8 (Ar—C), 166.2(C═O), 169.8 (C═N) p.p.m.

### Example 26

### Preparation of 7-chloro-3-(hydroxyamino)-5-phenyl-1,3-dihydro-2H-1,4-benzodiazepin-2-one

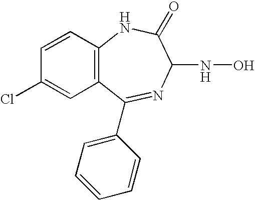

Following synthetic route A, the title compound was prepared and identified, as follows:

Yield: 44.0%.

Mpl. Formula: C₁₅H₁₂ClN₃O₂.

Mol. Weight: 301.7.

IR (KBr-disc) υ max: 3413, 3193, 3100, 2911, 2358, 1728, 1615, 1472, 1328, 1220, 1025, 693 cm⁻¹.

MS (APCI(+)): 301, 303 (M+1), 269, 271 (M+) m/z.

¹H NMR (DMSO-d₆) 300K δ: 10.67 (s, NH), 7.64-7.68 (dd, Ar—H, J=8.8 Hz), 7.46-7.50 (m, phenyl-5H), 7.28-7.32 (d, Ar—H, J=8.8 Hz), 7.21-7.23 (sd, Ar—H, J=2.5 Hz), 5.73 (s, OH), 4.81 (C3-H) ppm; ¹³C NMR (DMSO-d₆) 300K □: 75.6 (C₃), 121.6, 125.3 (2xC), 127.5, 128.9, 128.7 (2xC), 129.1, 130.2, 132.8, 137.2, 137.8 (Ar—C), 163.1 (C═N), 167.1 (C═O) p.p.m.

### Example 27

### Preparation of 3-(ethylamino)-7-chloro-5-phenyl-1,3-dihydro-2H-1,4-benzodiazepin-2-one

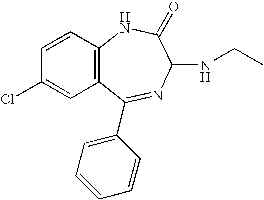

Following synthetic route A, the title compound was prepared and identified, as follows:

R_{f} (ether)=0.40.

Mol. Formula: C₁₇H₁₆ClN₃O.

Mol. Weight: 313.8.

IR (KBr-disc) υ max: 3430, 3121, 2977, 2855, 1654, 1607, 1478, 1320, 693 cm⁻¹.

MS (APCI(+)): 314, 315 (M+1), 296, 297 (M+), 269, 271 (M+) m/z.

¹H NMR (DMSO-d₆) 300K δ: 1.16-1.24 (t, 3H, J=7.1 Hz), 2.70-3.0 (m, —CH₂—), 4.34 (s, C3-H), 7.24-7.61 (m, Ar-8H), 11.19 (s, NH) p.p.m.

¹³C NMR (DMSO-d₆) 300K δ: 14.7 (CH₃), 42.4 (—CH₂—), 71.3 (C3), 123.4, 125.8 (2xC), 128.3, 129.7 (2xC), 130.6, 131.1, 136.3, 137.7, 166.5 (C═O), 169.0 (C═N) p.p.m.

### Example 28

### Preparation of 3-(propylamino)-7-chloro-5-phenyl-1,3-dihydro-2H-1,4-benzodiazepin-2-one

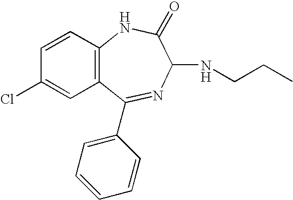

Following synthetic route A, the title compound was prepared and identified, as follows:

R_{f} (ether)=0.44.

Mol. Formula: C₁₈H₁₈ClN₃O

Mol. Weight: 327.8.

IR (KBr-disc) υ max: 3433, 3108, 2980, 2851, 1764, 1471 1315, 699 cm⁻¹.

MS (APCI(+)): 326, 327 (M+1), 308, 309 (M+), 269, 271 (M+) m/z.

¹H NMR (DMSO-d₆) 300K δ: 1.11-1.20 (t, 3H, J=7.0 Hz), 2.51-2.72 (m, 4H, —CH₂—), 4.45 (s, C3-H), 7.25-7.83 (m, Ar-8H), 11.12 (s, NH) p.p.m.

¹³C NMR (DMSO-d₆) 300K δ: 12.9 (CH₃), 34.0 & 42.4 (—CH₂—), 71.1 (C3), 123.4, 125.1 (2xC), 128.5, 129.9 (2xC), 130.0, 131.3, 136.4, 137.7 (Ar—C), 166.2 (C═O), 169.5 (C═N) p.p.m.

### Example 29

### Preparation of 3-(butylamino)-7-chloro-5-phenyl-1,3-dihydro-2H-1,4-benzodiazepin-2-one

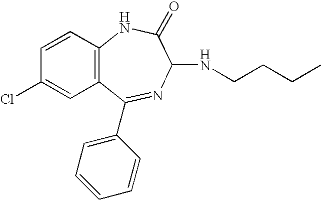

Following synthetic route B, the title compound was prepared and identified, as follows:

Yield: 25.0%.

R_{f} (ethylacetate)=0.54.

Mol. Formula: C₁₉H₂₀ClN₃O.

Mol. Weight: 341.8.

IR (KBr-disc) υ max: 3425, 3100, 2930, 2850, 1700, 1640, 1615, 1480, 1330, 1080 cm⁻¹. MS (APCI(+)): 342, 344 (M+1), 324, 326 (—H₂O), 269, 271 (M+) m/z.

¹H NMR (DMSO-d₆) 300K δ: 11.57 (s, NH), 9.69 (s, NH), 7.77 (dd, Ar—H, J=8.7 Hz), 7.60-7.49 (m, phenyl-5H), 7.42 (d, Ar—H, J=8.8 Hz), 7.30 (s, Ar—H), 5.12 (s, C3-H), 3.21 (m, —CH—), 3.02 (m, —CH—), 1.73 (m, —CH₂—), 1.39 (m, —CH₂—), 0.92 (t, CH₃, J=7.2, 7.4 Hz) p.p.m.

¹³C NMR (DMSO-d₆) 300K δ: 13.7 (CH₃), 21.9, 30.2, 45.2 (CH₂), 70.5 (C3), 121.9, 125.1 (2xC), 127.6, 128.2, 129.9 (2xC), 130.8, 131.3, 132.0, 136.8, 138.2 (Ar—C), 167.5(C═O), 168.1 (C═N) p.p.m.

### Example 30

### Preparation of 7-chloro-3-(cyclohexylamino)-5-phenyl-1,3-dihydro-2H-1,4-benzodiazepin-2-one

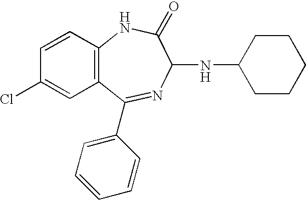

Following synthetic route A, the title compound was prepared and identified, as follows:

Yield: 33.0%.

Mol. Formula: C₂₁H₂₂ClN₃O.

Mol. Weight: 367.9.

IR (KBr-disc) υ max: 3403, 3092, 3033, 2927, 2863, 2358, 1700, 1623, 1474, 1322, 695 cm⁻¹.

MS. (APCI(+)): 368, 370 (M+1), 350, 352 (—H₂O), 269, 271 (M+) m/z.

¹H NMR (DMSO-d₆) 300K δ: 11.57 (s, NH), 9.58 (s, NH), 7.74-7.79 (dd, Ar—H, J=8.7 Hz), 7.43-7.63 (m, phenyl-5H), 7.40-7.43 (d, Ar—H, J=8.8 Hz), 7.30-7.31 (sd, Ar—H, J=2.4 Hz), 5.15 (s, C3-H), 1.07-2.22 (m, —CH₂—, 11H) p.p.m.

¹³C NMR (DMSO-d₆) 300K δ: 24.7, 25.3, 28.6, 29.8, 30.8 (—CH₂—), 54.6 (—CH—), 124.4 (2xC), 127.9, 128.0, 129.1, 130.3, 130.7, 131.9, 133.2, 137.8 (2xC), 137.9 (Ar—C), 165.7 (C═O), 167.8 (C═N) p.p.m.

The following were also synthesised:

### Example 31

### 7-chloro-3-(3,4-dimethylanilino)-5-phenyl-1,3-dihydro-2H-1,4-benzodiazepin-2-one

### Example 32

### 7-chloro-3-(piperidin-1-yl)-5-phenyl-1,3-dihydro-2H-1,4-benzodiazepin-2-one

### Example 33

### 7-chloro-3-(isopropylcyclohexylamino)-5-phenyl-1,3-dihydro-2H-1,4-benzodiazepin-2-one

### Example 34

### 7-chloro-3-(ethylcyclohexylamino)-5-phenyl-1,3-dihydro-2H-1,4-benzodiazepin-2-one

Biological Results—Examples 12 to 34

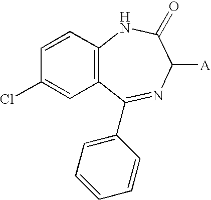

The amines were classified into five main series (0-4). Series 0 was composed of substituted anilines, heterocyclic and large bulky amines. Series 1 contained an unsubstituted aniline with 0, 1 or 2 carbon spacers between the amino group and the phenyl ring. Series 2 comprised the amines of series 1 but each with a N-methyl substituent. Series 3 contained various small groups and varying alkyl side chains. Series 4 was composed of analogues of Example 8 which was the most active from the previous screening result.

TABLE 2
Activity of further benzodiazepine analogues

| | | | | | | | |
|---|---|---|---|---|---|---|---|
| | | | | | MX | | IC₅₀ |
| | | Route | | | (M + 1) | Yield | CCK_{B} |
| Example | A | A/B | MW | FW | [m/z] | [%] | [μM] |

Series 0

| | | | | | | | |
|---|---|---|---|---|---|---|---|
| 13* | 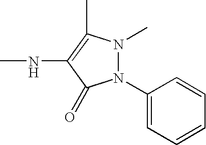 | A | 471 | C₂₆H₂₂ClN₅O₂ | 472 | 58 | 9.3 |
| 17* | 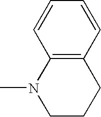 | A | 401 | C₂₄H₂₀ClN₃O | 402 | 66 | 7.2 |
| 18* | 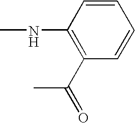 | B | 403 | C₂₃H₁₈ClN₃O | 404 | 33 | 8.4 |
| 19* | 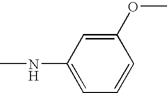 | A | 391 | C₂₂H₁₈ClN₃O₂ | 392 | 45 | 1.5 |
| 20* | 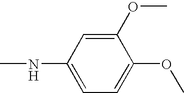 | A | 421 | C₂₃H₂₀ClN₃O₃ | 422 | 50 | 1.2 |
| 21* | 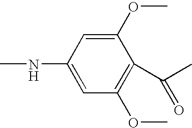 | B | 363 | C₂₅H₂₂ClN₃O₄ | 364 | 30 | 5.3 |
| 31* | 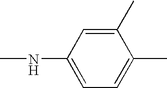 | A | 389 | C₂₃H₂₀ClN₃O | 390 | 58 | 0.92 |

Series 1

| | | | | | | | |
|---|---|---|---|---|---|---|---|
| 22* | 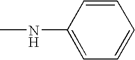 | A | 361 | C₂₁H₁₆ClN₃O | 362 | 80 | 4.1 |
| 23* | 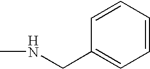 | A | 375 | C₂₂H₁₈ClN₃O | 376 | 62 | 6.0 |

Series 2

| | | | | | | | |
|---|---|---|---|---|---|---|---|
| 24* | 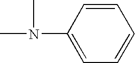 | A | 375 | C₂₂H₁₈ClN₃O | 376 | 39 | 0.15 |
| 25* | 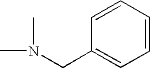 | A | 389 | C₂₃H₂₀ClN₃O | 390 | 36 | 2.9 |
| 26 | 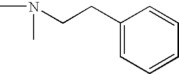 | A | 403 | C₂₄H₂₂ClN₃O | 404 | 36 | 9.8 |
| 28* | 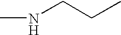 | A | 327 | C₁₈H₁₈ClN₃O | 328 | 39 | 8.9 |

Series 4

| | | | | | | | |
|---|---|---|---|---|---|---|---|
| 32 | 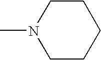 | B | 353 | C₂₀H₂₀ClN₃O | 354 | 21 | >15 |
| 30* | 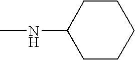 | A | 367 | C₂₁H₂₂ClN₃O | 368 | 33 | 10.1 |
| 33 | 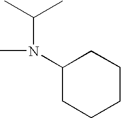 | A | 409 | C₂₄H₂₈ClN₃O | 410 | 31 | >15 |
| 34 | 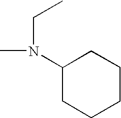 | A | 395 | C₂₃H₂₆ClN₃O | 396 | 30 | >15 |

* = fully characterised: TLC, IR, Mass-Spectrometry, ¹H & ¹³C NMR. The remainder were characterised by TLC and Mass-Spectrometry.

This further set of compounds were not as active as the first, IC₅₀s being much lower, mostly in the low micromolar range rather than the low nanomolar, as in the previous set of results, except Example 24.

To summarise, it can be concluded that large bulky and heterocyclic ring systems are likely to have little or no effect in displacing the radioactive ligand. Small aromatic, meta-substituted anilines exhibit good activity. Activity is lost by introducing an alkyl spacer unit, but is enhanced by the addition of an N-methyl group. From the results, compounds 31 and 24 are the most active from this series, at 920 & 150 nM respectively.

### Examples 35-56

### Synthesis and Biology of 3-(Substituted-aniline)-substituted Benzodiazepines

From Examples 12-34, it can be seen that compounds 31 and 24 demonstrated high binding affinity. By combining the common features of both compounds, it was possible to synthesise analogues of sub-general formula (IA). These would, potentially, show an enhanced binding for the CCK receptor. The selectivity of each analogue can be measured by comparing the ratio of CCK_{B} over CCK_{A} receptor subtypes.

No reaction yields are provided, since only a small portion of a mixture of the synthesised compounds was isolated. They were separated by preparative TLC (MP=ether), via the thionyl chloride route (Example 1, method A). Nitro-anilines were generally difficult to isolate, as the amine was difficult to remove through washing with dilute acid and water.

### Example 35

### Preparation of 7-chloro-3-(2-nitroanilino)-5-phenyl-1,3-dihydro-2H-1,4-benzodiazepin-2-one

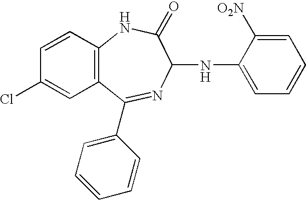

Oxazepam (0.1 g, 3.5×10⁻⁴ mol) was treated with thionyl chloride (4 Eq, 0.1 ml) and heated to 60° C. for 1.5 hours. The resulting intermediate, yellow solid, was washed with dry diethyl ether (twice) to remove any excess thionyl cholride. The appropriate substituted aniline 2.5 Eq, 1.1×10⁻³ mol), with TEA (drops) was added with dry DCM (15 ml) and refluxed for two hours. The organic phase was washed with hydrochloric acid (pH 4.0-5.0) and dried over sodium sulphate. Excess DCM was removed and preparative TLC (MP: ether, 6% Methanol in ether) isolated the desired product.

R_{f} (ether)=0.30.

Mol. Formula: C₂₁H₁₅ClN₄O₂.

Mol. Weight: 390.8.

IR (KBr-disc) υmax: 3349, 3279, 1702, 1590, 1527, 1469, 1316, 1297, 1114, 830, 693 cm⁻¹.

MS (APCI(+)): 391, 393 (M+1), 269, 271 (M+) m/z.

¹H NMR (DMSO-d₆) 300K δ 5.19-5.22 (d, C3-H, J=7.0 Hz), 6.82-6.86 (d, Ar—H, J=9.2 Hz), 7.10-7.18 (m, Ar-2H), 7.33 (s, Ar—H), 7.44-7.55 (m, phenyl-5H), 7.70-7.75 (dd, Ar—H, J=8.8 Hz), 7.09-8.02 (d, Ar—H, J=9.3 Hz), 7.04-7.07 (d, Ar—H, J=7.1 Hz), 11.15 (s, NH) p.p.m.

¹³C NMR (DMSO-d6) 300K δ 70.7 (C3), 113.2, 124.1, 126.3, 127.2 (2xC), 128.9, 129.9, 130.3, 131.3, 132.6, 137.5, 138.2 (2xC), 138.6, 153.2 (2xC), 166.8 (C═O), 167.7 (C═N) p.p.m.

### Example 39

### Preparation of 7-chloro-3-(3-chloroanilino)-5-phenyl-1,3-dihydro-2H-1,4-benzodiazepin-2-one

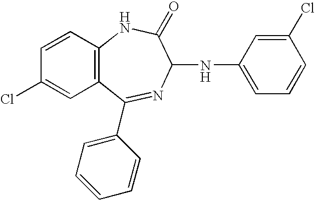

R_{f} (ether)=0.31.

Mol. Formula: C₂₁H₁₅Cl₂N₃O.

Mol. Weight: 396.3.

IR (KBr-disc) υ max: 3438, 2919, 2856, 2362, 2338, 1653, 1594, 1318, 1014, 671 cm⁻¹.

MS (APCI(+)): 396, 397, 398 (M+1), 378, 379, 380 (—H₂O), 269, 271 (M+) m/z.

¹H NMR (DMSO-d₆) 300K δ: 5.05 (s, C3-H), 6.80-6.60 (d, Ar—H, J=8.5 Hz), 6.64-6.69 (d, Ar—H, J=8.0 Hz), 6.80 (s, Ar—H), 7.05-7.11 (t, Ar—H, J=8.1, 8.1 Hz), 7.32 (s, Ar—H), 7.33-7.35 (d, Ar—H, J=8.7 Hz), 7.43-7.53 (m, phenyl-5H), 7.68-7.73 (dd, Ar—H, J=8.8 Hz), 11.06 (s, NH) p.p.m.

¹³C NMR (DMSO-d₆) 300K δ: 67.9 (C3), 117.5, 118.1, 122.6, 122.8, 125.8 (2xC), 128.4, 128.7, 130.1, 130.5 (2xC), 130.8, 130.9, 132.0, 133.5, 136.1, 137.2, 142.5 (Ar—C), 164.2 (C═O), 168.1 (C═N) p.p.m.

### Example 43

### Preparation of 7-chloro-3-(4-methoxyanilino)-5-phenyl-1,3-dihydro-2H-1,4-benzodiazepin-2-one

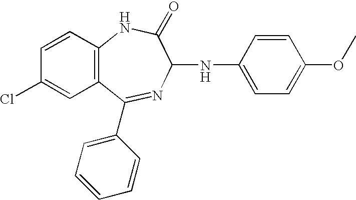

R_{f} (ether)=0.38

Mol. Formula: C₂₂H₁₈ClN₃O₂.

Mol. Weight: 391.9.

IR (KBr-disc) υ max: 3426, 3193, 3058, 2935, 1687, 1519, 1476, 1320, 1220, 698cm⁻¹.

MS (APCI(+)): 392, 394 (M+1), 374, 376 (—H₂O), 269, 271 (M+) m/z.

¹H NMR (DMSO-d₆) 300K δ: 3.77 (s, OCH₃) 4.80 (s, C3-H), 7.00-7.05 (d, Ar-2H, J=7.8 Hz), 7.28-7.31 (d, Ar—H, J=7.7 Hz), 7.32-7.36 (d, Ar—H, J=7.9 Hz), 7.46-7.55 (m, phenyl-5H), 7.63-7.68 (dd, Ar—H, J=8.8 Hz), 10.16 (s, NH), 10.85 (s, NH) p.p.m.

¹³C NMR (DMSO-d₆) 300K δ: 53.3 (OCH₃), 68.1 (C3), 116.3 (2xC), 117.2 (2xC) 122.3, 124.5 (2xC), 124.9, 126.6, 126.9, 129.8 (2xC), 129.9, 130.6, 137.0, 137.4, 139.7, 153.6, 165.3 (C═O), 167.1 (C═N) p.p.m.

### Example 51

### Preparation of 7-chloro-3-(2,3-dimethylanilino)-5-phenyl-1,3-dihydro-2H-1,4-benzodiazepin-2-one

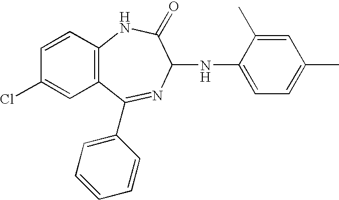

R_{f} (ether)=0.44.

Mol. Formula: C₂₃H₂₀ClN₃O.

Mol. Weight: 389.9.

IR (KBr-disc) υ max: 3436, 3182, 2919, 2618, 1690, 1606, 1473, 1222, 1147 cm⁻¹.

MS (APCI(+)): 390, 392, (M+1), 269, 271 (M+) m/z.

¹H NMR (DMSO-d₆) 300K δ: 2.32 (s, CH₃), 2.78 (s, CH₃), 4.80 (s, C3-H), 7.08-7.18 (m, Ar-2H), 7.22-7.23 (s,d Ar—H, J=2.5 Hz), 7.28-7.31 (d, Ar—H, J=8.5 Hz), 7.32-7.35 (d, Ar—H, J=7.5 Hz), 7.46-7.53 (m, phenyl-5H), 7.63-7.68 (dd, Ar—H, J=8.7, 8.8 Hz), 10.18 (s, NH), 10.84 (s, NH) p.p.m.

¹³C NMR (DMSO-d₆) 300K δ: 17.5 (CH₃), 21.0 (CH₃), 83.2 (C3), 123.8 (2xC), 125.8, 127.2, 127.8, 128.1, 128.7, 129.0 (2xC), 129.4, 129.9, 131.2, 132.3, 132.4, 132.5, 138.2, 138.4 (Ar—C), 163.5 (C═O), 170.1 (C═N) p.p.m.

### Example 55

### Preparation of 7-chloro-3-(3-dimethylanilino)-5-phenyl-1,3-dihydro-2H-1,4-benzodiazepin-2-one

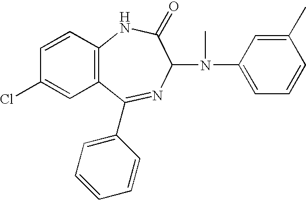

R_{f} (ether)=0.66.

Mol. Formula: C₂₃H₂₀ClN₃O.

Mol. Weight: 389.9.

IR (KBr-disc) υ max: 3420, 2925, 1700, 1600, 1481, 1320, 1121, 699 cm⁻¹.

MS (APCI(+)): 390, 392, (M+1), 269, 271 (M+) m/z.

¹H NMR (DMSO-d₆) 300K δ: 2.22 (s, CH₃), 3.25 (s, N—CH₃), 5.61 (s, C3-H), 7.00-7.06 (t, Ar—H, J=7.8, 7.9 Hz), 7.23 (s, Ar—H), 7.22-7.30 (d, Ar—H, J=7.4 Hz), 7.31 (s, Ar—H), 7.37-7.40 (d, Ar—H, J=8.7 Hz), 7.49-7.56 (m, phenyl-5H), 7.64-7.66 (dd, Ar—H, J=8.8 Hz), 7.69-7.74 (dd, Ar—H, J=8.7 Hz), 10.89 (s, NH) p.p.m.

¹³C NMR (DMSO-d₆) 300K δ: 22.0 (CH₃), 58.6 (N—CH₃), 71.8 (C3), 122.3, 124.6 (2xC), 125.2, 125.7, 127.0, 127.6, 128.6, 128.1, 128.9, 129.0, 129.3, 129.8 127.2, 127.8, 128.1, 128.7, 129.0, 129.3, 129.8 (2xC), 129.9, 138.4, 138.4, 138.6, 149.5 (Ar—C), 165.1 (C═O), 169.4 (C═N) p.p.m.

TABLE 3
Examples 35 to 56 Biological Results

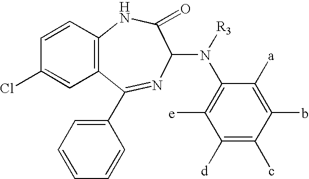

| | | | | | | | | MS | IC₅₀ | | Ratio |
|---|---|---|---|---|---|---|---|---|---|---|---|
| | | | | | | | | (M + 1) | CCK_{B} | CCK_{A} | CCK_{B}: |
| Example | R₃ | a | b | c | d | e | R_{f} | [m/z] | [μM] | [μM] | CCK_{A} |
| 35* | H | NO₂ | H | H | H | H | 0.45 | 407 | 7.5 | >20 | — |
| 36 | H | H | NO₂ | H | H | H | 0.21 | 407 | 6 | 0.011 | 545 |
| 37 | H | H | H | NO₂ | H | H | 0.37 | 407 | 8.5 | >20 | — |
| 38 | H | Cl | H | H | H | H | 0.44 | 396 | 9.3 | >20 | — |
| 39* | H | H | Cl | H | H | H | 0.40 | 396 | 6.2 | 0.27 | 23 |
| 40 | H | H | H | Cl | H | H | 0.38 | 396 | 2.9 | 20 | — |
| 41 | H | OMe | H | H | H | H | 0.37 | 392 | 1.5 | >20 | — |
| 42* | H | H | OMe | H | H | H | 0.30 | 392 | 4.5 | 0.010 | 450 |
| 43* | H | H | H | OMe | H | H | 0.34 | 392 | >10 | 20 | — |
| 44 | H | CH₃ | H | H | H | H | 0.38 | 376 | 7.1 | 10 | +113 |
| 45 | H | H | CH₃ | H | H | H | 0.40 | 376 | 3.8 | 0.011 | 380 |
| 46 | H | H | H | CH₃ | H | H | 0.29 | 376 | 4.6 | 0.29 | 16 |
| 47 | H | CH₃ | H | H | H | CH₃ | 0.48 | 390 | >10 | 10 | — |
| 48 | H | H | CH₃ | CH₃ | H H | 0.25 | 390 | 0.92 | 0.015 | 61 | |
| 49 | H | CH₃ | CH₃ | H | H | H | 0.36 | 390 | 6 | 0.018 | 333 |
| 50 | H | H | CH₃ | H | CH₃ | H | 0.20 | 390 | 2.9 | 0.009 | 322 |
| 51* | H | CH₃ | H | CH₃ | H | H | 0.42 | 390 | 6.1 | 0.39 | 16 |
| 52 | H | CH₃ | H | H | CH₃ | H | 0.48 | 390 | 6.0 | 1.0 | 6 |
| 53* | H | H | H | H | H | H | 0.37 | 362 | 4.1 | 0.24 | 17 |
| 54* | CH₃ | H | H | H | H | H | 0.57 | 376 | 0.15 | 0.014 | 11 |
| 55* | CH₃ | H | CH₃ | H | H | H | 0.52 | 390 | 0.07 | 0.008 | 9 |
| 56 | C₂H₅ | H | CH₃ | H | H | H | 0.60 | 404 | 0.65 | 0.33 | 2 |

*Fully characterised

The entire combination of substituted anilines was successfully synthesised and separated, based on the two initial lead compounds (Examples 24 and 31). The results, from Table 3 show that several of the synthesised compounds are exceedingly potent towards the CCK_{A} receptor, the binding to the CCK_{A+} receptor generally being less. Nitro-anilines displayed weak CCK_{B} binding. However, Example 36, a meta-nitro-aniline showed exceptional CCK_{A} binding at IC₅₀=11 nM, whilst the ortho & para groups were inactive (IC₅₀=>20 μM). This same result was observed for the m-chloro-anilines (Example 39, IC₅₀=270 nM), the m-methoxyaniline (Example 42, IC₅₀=10 nM) and the m-toluidine (Example 45, IC₅₀=11 nM). Dimethylanilines were equally active, when at least one group was at the meta-position (Examples 48, 49 and 50), whilst the N-methylaniline (Example 54) had an IC₅₀ value of 14 nM.

Example 55 was the most active compound in the series for both receptors, at 70 & 8 nM for the CCK_{B} & CCK_{A} receptor subtypes, respectively. It can be deduced that removing the urea functionality produces analogues that are less potent towards the CCK_{B} receptor. However, activity is in the nanomolar range towards the CCK_{A} receptor, especially for the meta-positioned substitutents. Selectivity is up to 550 for the A receptor subtype, but most important these anilinobenzodiazepine salts are freely soluble in DMSO and water.

### Examples 57 to 63

### Synthesis and Biology of N-alkylated Benzodiazepines

First, oxazepam was alkylated in accordance with the method of Description 2: A 50% suspension of NaH in mineral oil (0.06 mol) was added in drops to a solution of oxazepam ( 0.05 mol) in dry DMF (100 ml). After stirring for 15 mins at RT, the alkylating agent (0.06 mol) was added in drops to the mixture, with ice cooling. The solution was stirred for additional 30-45 mins at RT. For workup: Water was added (75 ml) and the suspension was added to ethylacetate (75 ml). The extract was washed with brine (100 ml×2), dried over sodium sulphate, with the solvent evaporated. Column chromatography, with ether/petrolether 1:2 as the eluent.

Mass spectrometric analysis of the alkylated products was achieved using the negative mode of the APCI instrument, since the positive mode failed to detect the M+1 product peaks. All synthesised compounds were screened on the CCK_{B} receptor subtype.

### Example 57

### Preparation of 7-chloro-1-(3,3-dimethyl-2-oxobutyl)-3-hydroxy-5-phenyl-1,3-dihydro-2H-1,4-benzodiazepin-2-one

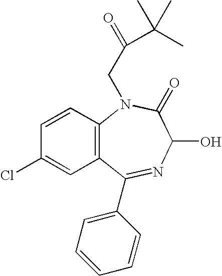

Yield: 81%.

R_{f} (ether/petrolether 1:2)=0.51

Mol. Weight: 384.9.

Mol. Formula: C₂₁H₂₁ClN₂O₃.

MS (APCI(−)): 383, 385 (M−1), 285, 287 (M+) m/z.

IR (KBr-disc) υ max: 3450,2933, 2358, 1710, 1677, 1596, 1482, 1322, 1131 & 693 cm⁻¹.

¹H NMR (CDCl₃) 300K δ: 1.23 (s, (CH₃)₃), 4.81 (s, C3-H), 5.04-5.12 (m, —CH₂—), 7.05-7.67 (m, Ar-9H) p.p.m.

¹³C NMR (DMSO-d₆) 300K δ: 26.3 (CH₃)₃), 43.5 ((CCH₃)₃), 53.2 (CH₂), 82.0 (C3), 122.9, 128.3 (2xC), 128.4, 129.6, 129.8 (2xC), 130.4, 130.8, 131.9, 137.4, 140.1 (Ar—C), 155.3 (C═O), 166.9 (C═N), 169.4 (C═O) p.p.m.

### Example 58

### Preparation of 7-chloro-3-hydroxy-5-phenyl-1-prop-2-ynyl-1,3-dihydro-2H-1,4-benzodiazepin-2-one

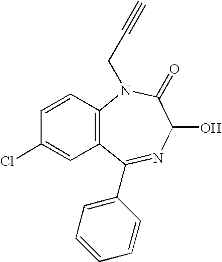

Yield: 67%.

R_{f} (ether/petrolether 1:2)=0.38

Mol. Weight: 324.8.

Mol. Formula: C₁₈H₁₃ClN₂O₂.

MS (APCI(−)): 323, 325 (M−1), 284, 286 (M+) m/z.

IR (KBr-disc) υ max: 3418, 3291, 3225, 2923, 1700, 1634, 1478, 1415, 1324, 1131, 1002 & 695 cm⁻¹.

¹H NMR (CDCl₃) 300K δ: 2.10-2.34 (t, CH, J=24.7, 25.0 Hz), 4.51-4.66 (m, —CH₂—), 5.04 (C3), 7.21-7.63 (Ar—H) p.p.m.

¹³C NMR ((CDCl₃)) 300K δ: 37.0 (—CH₂—), 73.5, 75.19 (CH), 86.6 (C3), 123.4, 128.3 (2xC), 128.3, 129.4 (2xC), 130.3, 130.7, 131.1, 132.1, 137.1, 139.5 (Ar—H), 164.6 (C═O), 166.1 (C═N) p.p.m.

The following Examples were also prepared:

### Example 59

### 7-chloro-3-hydroxy-5-phenyl-1-benzyl-1,3-dihydro-2H-1,4-benzodiazepin-2-one

### Example 60

### 7-chloro-3-hydroxy-5-phenyl-1-allyl-1,3-dihydro-2H-1,4-benzodiazepin-2-one

### Example 61

### 7-chloro-3-hydroxy-5-phenyl-1-ethoxycarbonyl-1,3-dihydro-2H-1,4-benzodiazepin-2-one

### Example 62

### 7-chloro-3-hydroxy-5-phenyl-1-phenacyl-1,3-dihydro-2H-1,4-benzodiazepin-2-one

### Example 63

### 7-chloro-3-hydroxy-5-phenyl-1-(2-(4-morpholino)ethyl)-1,3-dihydro-2H-1,4-benzodiazepin-2-one

TABLE 4

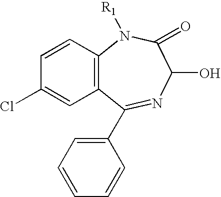

Structure and activity of alkylated Oxazepam derivatives

| | | | | | | MS | | IC₅₀ |
|---|---|---|---|---|---|---|---|---|
| | Alkylating | | | | | (M − 1) | Yield | CCK_{B} |
| Example | agent | Rₜ | MF | MW | R_{f} | [m/z] | [%] | [nM] |
| 57* | Trimethyl- acetyl chloride | 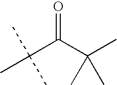 | C₂₀H₁₉ClN₂O₃ | 370 | 0.51 | 369 | 81 | 190 |
| 58* | Propargyl bromide | 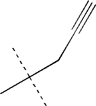 | C₁₈H₁₃ClN₂O₂ | 324 | 0.38 | 323 | 67 | 960 |
| 59 | Benzyl chloride | 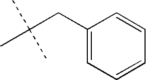 | C₂₂H₁₇ClN₂O₃ | 376 | 0.42 | 375 | 40 | 760 |
| 60 | Allyl bromide | 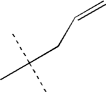 | C₁₈H₁₅ClN₂O₂ | 326 | 0.35 | 325 | 68 | 980 |
| 61 | Ethyl chloro- formate | 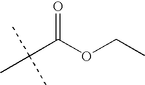 | C₁₈H₁₁₅ClN₂O₄ | 358 | 0.47 | 357 | 29 | 690 |
| 62 | Phenacyl chloride | 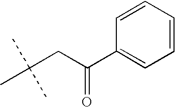 | C₂₃H₁₇ClN₂O₃ | 404 | 0.44 | 403 | 25 | 200 |
| 63 | 4-(2-chloro- ethyl)morph- oline HCl | 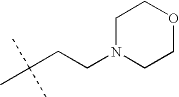 | — | — | — | — | — | — |

### Example 64

### Preparation of 7-chloro-1-(3,3-dimethyl-2-oxobutyl)-3-(3-dimethylanilino)-5-phenyl-1,3-dihydro-2H-1,4-benzodiazepin-2-one

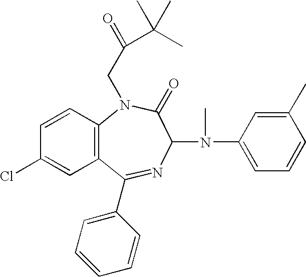

A suspension of compound 3.6.2 (7-chloro-1-(3,3-dimethyl-2-oxobutyl)-3-hydroxy-5-phenyl-1,3-dihydro-2H-1,4-benzodiazepin-2-one) (2 g, 7.1 mmols) and NBS (N-bromosuccinimide) (1.53 g, 8.6 mmols) in carbon tetrachloride (80 ml) was stirred at ambient temperature for 20 mins. Trifluoroacetic acid (70 mg, 0.6 mmols) was added and then mixture was vigorously stirred and heated, under reflux, for 1.5 hours. The hot solution was cooled, separated from the yellow, sticky precipitate by decantation. The residue was washed with carbon tetrachloride (2×30 ml). The combined solution was evaporated to dryness to give the bromo-intermediate in 91% yield.

2 g of this material, with N-methyl-m-toluidine (2.5 Eq, 0.95 ml) was stirred in dry DCM (30 ml), with a few drops of TEA for 15 mins. The mixture was refluxed for 2 hours. Afterwards the organic phase was washed with hydrochloric acid (pH 4.0-5.0) and dried over sodium sulphate and the residue purified by preparative chromatography (MP: ether/petrolether) to give a yellow powder.

Yield: 4.9%.

R_{f} (1:2)=0.54.

Mol. Weight: 488.0.

Mol. Formula: C₂₉H₃₀ClN₃O₂.

MS (APCI(−)): 487, 389 (M−1), 366, 368 (M+) m/z.

IR (KBr disc) υ max: 3460, 3325, 2823, 2418, 1721, 1643, 1602, 1571, 1266 & 697 cm⁻¹.

¹H NMR (CDCl₃) 300K δ: 1.21 (s, (CH₃)₃), 2.19 (CH₃), 3.24 (N—CH₃), 4.88 (s, C3-H), 5.06-5.15 (m, —CH₂—), 7.01-7.42 (m, Ar-5H), 7.49-7.77 (m, Ar-7H) p.p.m.

Biological Activity

The in vitro activity of Example 64 produced an IC₅₀, for both the CCK_{B} & CCK_{A} receptors at around 8 & 24 nM, respectively. However, Example 64 has the potential of demonstrating better bio-availability than prior art compounds, with the nitrogen being easily protonated for oral administration. Example 64 was evaluated as a racemic mixture; one isomer may be more potent than the other and selective over either receptor subtype.

What is claimed is:

1. A compound of formula (I) 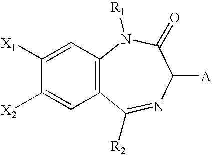
wherein each of X₁, X₂, and R₂ is independently selected from hydrogen, a halogen, a substituted or unsubstituted cyclic and heterocyclic moiety, substituted or unsubstituted, linear or branched alkyl, alkyloxy, alkylcarbonyl, alkyloxycarbonyl, alkenyl, alkenyloxy, alkenylcarbonyl, alkenyloxycarbonyl, alkynyl, alkynyloxy, alkynylcarbonyl, alkynyloxycarbonyl, aryl, benzyl, aryloxy, arylcarbonyl, aryloxycarbonyl and sulphur equivalents of said oxy, carbonyl and oxycarbonyl moieties, and a nitrogen containing functional group,
R₁ is selected from hydrogen, a halogen, a substituted or unsubstituted cyclic and heterocyclic moiety, substituted or unsubstituted, linear or branched alkyl, alkylcarbonyl, alkyloxycarbonyl, alkenyl, alkenylcarbonyl, alkenyloxycarbonyl, alkynyl, alkynylcarbonyl, alkynyloxycarbonyl, aryl, benzyl, arylcarbonyl, aryloxycarbonyl and sulphur equivalents of said, carbonyl and oxycarbonyl moieties and
A is selected from hydrogen, hydroxyl, a halogen, a nitrogen-containing heterocycle linked to the diazepine moiety via nitrogen and 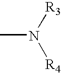
wherein R₃ and R₄ are independently selected from hydrogen, a halogen, a substituted or unsubstituted cyclic and heterocyclic moiety, substituted or unsubstituted, linear or branched alkyl, alkylcarbonyl, alkyloxycarbonyl, alkenyl, alkenylcarbonyl, alkenyloxycarbonyl, alkynyl, alkynylcarbonyl, alkynyloxycarbonyl, aryl, benzyl, arylcarbonyl, aryloxycarbonyl and sulphur equivalents of said, carbonyl and oxycarbonyl moieties and wherein if A is OH, then R₁ is selected from a substituted or unsubstituted cyclic and heterocyclic moiety, substituted or unsubstituted, linear or branched alkyl, alkylcarbonyl, alkyloxycarbonyl, alkenyl, alkenylcarbonyl, alkenyloxycarbonyl, alkynyl, alkynylcarbonyl, alkynyloxycarbonyl, aryl, benzyl, arylcarbonyl, aryloxycarbonyl and sulphur equivalents of said, carbonyl and oxycarbonyl moieties

2. A compound as claimed in claim 1, wherein said alkyl-containing moieties are C₁-C₁₂.

3. A compound as claimed in claim 1, wherein said alkenyl- and said alkynyl-containing moieties are C₂-C₁₂.

4. A compound as claimed in claim 1, wherein said aryl moiety is substituted or unsubstituted phenyl, napthyl or indolyl.

5. A compound as claimed in claim 4, wherein said aryl moiety is selected from m-substituted phenyl, indol-2-yl and indol-3-yl.

6. A compound as claimed in claim 1, wherein said substituents for said heterocyclic, alkyl, alkenyl, alkynyl and aryl moieties are selected from halo, amino, nitro, hydroxy, alkoxy and cyano moieties.

7. A compound as claimed in claim 1, wherein said heterocyclic moiety is a monocyclic or bicyclic ring comprising at least one of oxygen, sulphur and nitrogen.

8. A compound as claimed in claim 1, wherein said cyclic alkyl moiety is a 3 to 7 membered ring and said cyclic alkenyl and alkynyl moieties are 4 to 7 membered rings.

9. A compound as claimed in claim 1, wherin X₁ and X₂ are independently selected from hydrogen, C₁₋₄ alkyl, halogen, nitro, amino and C₁₋₄ alkoxy.

10. A compound as claimed in claim 1, wherein R₁ is selected from hydrogen, C₁₋₄ alkyl, benzyl, alkylcarbonyl, alkyloxycarbonyl, arylcarbonyl, alkenyl, alkynyl alkylcarbonylmethyl, arylcarbonylmethyl and morpholinylalkyl.

11. A compound as claimed in claim 10, wherein R₁ is selected from phenylmethyl, ^{t}butylcarbonyl, propargyl, allyl, C₁₋₄ alkyloxycarbonyl, phenylcarbonylmethyl and morpholinyl C₁₋₄ alkyl.

12. A compound as claimed in claim 1, wherein R₂ is phenyl or cyclohexyl.

13. A compound as claimed in claim 1, wherein A is a substituted nitrogen-containing heterocycle, selected from morpholinyl, pyrazolyl, piperazinyl, piperidinyl, quinolinyl, 3,4-dihydroquinolin-1(2H)-yl, and indolyl.

14. A compound as claimed in claim 1, wherein R₃ and R₄ are independently selected from hydrogen, C₁₋₄ alkyl, (CH₂)ₙC₁₋₆ alkyl, (CH₂)ₙC₃₋₆ cycloalkyl, pyrenyl, tetrahydronaphthyl, morpholinyl, 1-phenyl-pyrazol-2-yl, tetrahydroquinolyl and phenyl, and wherein n is 0,1 or 2.

15. A compound as claimed in claim 14, wherein R₃ or R₄ is phenylmono-di-or tri-substituted with one or more functional groups selected from halogen, C₁₋₄ alkyl, C₁₋₄ alkyloxy, C₁₋₄ alkylcarbony and nitro.

16. A compound as claimed in claim 15, wherein said phenyl is substituted with at least one of methyl, methoxy, chloro and acetyl.

17. A compound as claimed in claim 14, wherein said phenyl is at least meta-substituted.

18. A compound as claimed in claim 14, wherein one of R₃ and R₄ is hydrogen, methyl, ethyl, isopropyl or propyl and the other of R₃ and R₄ is substituted or unsubstituted phenyl or cyclohexyl.

19. A compound as claimed in claim 1, wherein A is a substituted aniline.

20. A method of producing a compound of Formula (1), comprising the stepsof:
(i) providing a leaving group L at the C-3 position of compound (II) in which B is hydrogen or hydroxyl to give compound(III),
(ii) displacing said leaving group with an amino moiety A to give compound (I), 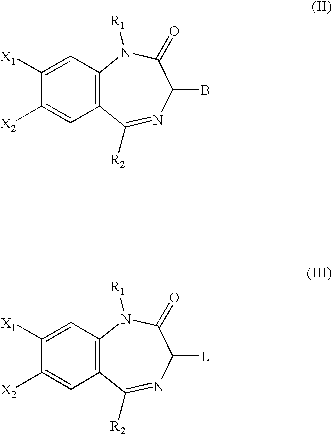
wherein R₁, R₂, X₁ and X₂ are as defined in claim 1 and A is selected from a nitrogen-containing heterocycle linked to the diazepine moiety via nitrogen and 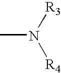
where R₃ and R₄ are as defined in claim 1.

21. The method of claim 20, wherein leaving group L is selected from chloro, bromo or iodo.

22. The method as claimed in claim 20, wherein step (i) is achieved by free radical substitution, when B is H.

23. The method as claimed in claim 20, wherein step (i) is achieved by nucleophilic substitution, when B is OH.

24. The method as claimed in claim 20, wherein step (i) is a two step procedure.

25. The method as claimed in claim 20, including a step of separating optical isomers.

26. (canceled)

27. (canceled)

28. A method of treatment of a mammal afflicted with a CCK-receptor mediated condition, or prophylaxis in a mammal at risk of a CCK-receptor mediated condition comprising administering a therapeutically effective amount of a compound as claimed in claim 1.

29. (canceled)

30. The method of claim 28, wherein said CCK-receptor mediated condition is a GI disorder, a CNS disorder caused by CCK interaction with dopamine, another CNS disorder; oncologic disorder, disorder of appetite regulatory systems; Zollinger-Ellison syndrome; antral G cell hyperplasia; or pain.

31. The method of claim 30, wherein said GI disorder is selected from irritable bowel syndrome, gastro-oesophageal reflux disease or ulcers, excess pancreatic or gastric secretion, acute pancreitis, or motility disorders; said CNS disorder is selected from neuroleptic disorders, tardive dyskinesia, Parkinson's disease, psychosis or Gilles de la Tourette syndrome, said another CNS disorder is selected from anxiety disorders and panic disorders and said oncologic disorder is selected from small cell adenocarcinomas and primary tumours of the central nervous system glial and neuronal cells.

32. A method of inhibiting CCK receptor activity comprising contacting a composition comprising a CCK receptor with the compound of claim 1.

33. The method of claim 32, wherein said ligand is a selective CCK1 or CCK2 ligand.

34. A composition for the treatment or prophylaxis of a CCK-receptor mediated condition comprising the compound of claim 1 and a pharmaceutically acceptable carrier.
